(19) United States
(12) Patent Application Publication
(10) Pub. No.: US 2026/0355357 A1
(43) Pub. Date: Nov. 12, 2026

(54) DIAGNOSTIC AGENTS AND USES THEREOF

(71) Applicant: HADASIT MEDICAL RESEARCH SERVICES AND DEVELOPMENT LTD., Jerusalem (IL)

(72) Inventors: Rachel KATZ-BRULL, Jerusalem (IL); Jacob SOSNA, Jerusalem (IL); Moshe John GOMORI, Jerusalem (IL)

(73) Assignee: HADASIT MEDICAL RESEARCH SERVICES AND DEVELOPMENT LTD., Jerusalem (IL)

(21) Appl. No.: 19/713,388

(22) Filed: Jun. 18, 2026

Related U.S. Application Data
(63) Continuation-in-part of application No. PCT/IL2024/051204, filed on Dec. 19, 2024.
(60) Provisional application No. 63/613,391, filed on Dec. 21, 2023.

(51) Int. Cl. A61K 49/10

(52) U.S. Cl. CPC A61K 49/10

(57) ABSTRACT

The present disclosure relates to 2-deoxy-D-glucose (2DG) compounds comprising one or more isotopically substituted hydrogen atoms, uses and methods thereof.

### CROSS-REFERENCE TO RELATED APPLICATIONS

This application is a continuation-in-part of International Patent Application No. PCT/IL2024/051204, filed on Dec. 19, 2024, which claims priority to U.S. Application No. 63/613,391, filed Dec. 21, 2023. The entire contents of these applications are incorporated herein by reference in their entirety.

### TECHNOLOGICAL FIELD

The present disclosure relates to diagnostic agents and methods for their use.

### BACKGROUND ART

References considered to be relevant as background to the presently disclosed subject matter are listed below:

[1] H M De Feyter, et al., Deuterium metabolic imaging (DMI) for MRI-based 3D mapping of metabolism in vivo Science Advances, 4, 2018.

Acknowledgement of the above references herein is not to be inferred as meaning that these are in any way relevant to the patentability of the presently disclosed subject matter.

### BACKGROUND

Deuterium Metabolic Imaging (DMI) is used to map the distribution of a deuterium-labelled metabolite and its metabolic fates in a biological sample or subject [1]. Deuterium-labelled glucose ([6,6-D₂]D-glucose) has been used together with DMI to follow metabolic fates of glucose such as lactate and glutamine/glutamate [1].

### GENERAL DESCRIPTION

The present disclosure provides in accordance with some aspects, a 2-deoxy-D-glucose (2DG) compound comprising one or more isotopically substituted hydrogen atoms for use in a method of acquiring at least one magnetic resonance (MR) spectrum and/or at least one MR image.

The present disclosure provides in accordance with some other aspects, a 2DG compound for use in a method of acquiring at least one magnetic resonance (MR) spectrum and/or at least one MR image, wherein the compound is represented by Formula (I) or a pharmaceutically acceptable salt, solvate, hydrate, any tautomer thereof, any isomer thereof, any metabolite thereof or physiologically functional derivative thereof, wherein at least one of X₁, X₂, X₃, X₄, X₅, X₆, X₇, X₈ is ²H (D).

The present disclosure provides in accordance with some other aspects, a 2DG compound for use in a method of acquiring at least one magnetic resonance (MR) spectrum and/or at least one MR image, wherein the compound is represented by Formula (II) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

The present disclosure provides in accordance with some further aspects, a 2DG compound for use in a method of diagnosing a subject, wherein the 2DG compound comprising one or more isotopically substituted hydrogen atoms.

The present disclosure provides in accordance with yet some aspects, a 2DG compound for use in a method of diagnosing a disease in a subject, wherein the 2DG compound comprising one or more isotopically substituted hydrogen atoms.

The present disclosure provides in accordance with yet some other aspects, a 2DG compound for use in detecting a metabolite of said compound.

The present disclosure provides in accordance with yet some further aspects, a 2DG compound for use in (i) a method of monitoring a disease state in said subject and/or (ii) a method of determining a site of a disease in said subject, wherein the 2DG compound comprising one or more isotopically substituted hydrogen atoms.

The present disclosure provides in accordance with yet some further aspects, a 2DG compound for use in a method of distinguishing between healthy and abnormal tissues or organs and/or distinguishing or differentiating between malignant and benign tumors, wherein the 2DG compound comprising one or more isotopically substituted hydrogen atoms.

The present disclosure provides in accordance with yet some aspects, a diagnostic formulation comprising at least one 2DG compound comprising one or more isotopically substituted hydrogen atoms.

The present disclosure provides in accordance with yet some aspects, a kit comprising a formulation as described herein, and instructions for use thereof.

The present disclosure provides in accordance with yet some aspects, a method of imaging a subject, the method comprising monitoring a signal from a subject using an MR imaging method, the subject having been administered at least one 2DG compound to thereby acquire at least one MR image, wherein said 2DG compound is deuterium-labeled 2DG comprising one or more isotopically substituted hydrogen atoms.

The present disclosure provides in accordance with yet some aspects, a method for diagnosis of a disease or condition in a subject, said method comprising (i) administering to the subject a diagnostically effective amount of at least one 2DG compound and (ii) imaging and/or acquiring a spectrum of said subject or a body region of the subject to thereby identify body regions in which said compound, or any metabolite thereof has been localized, wherein said 2DG compound is deuterium-labeled 2DG, comprising one or more isotopically substituted hydrogen atoms.

The present disclosure provides in accordance with yet some aspects, a method for monitoring a disease state in a subject, the method comprising (i) provided a compound is administered to the subject; (ii) obtaining an image from the subject's body or any one or more regions thereof, whereby obtaining at least one imaging parameter indicative of the disease or disorder state; and (iii) comparing said at least one imaging parameter to at least one parameter obtained from said subject at an earlier timepoint; wherein the comparison enables determining the progression of the disease or disorder state; and wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI) or a pharmaceutically acceptable salt, solvate, hydrate, any isomer thereof, any metabolite thereof or physiologically functional derivative thereof, or (f) a combination thereof.

The present disclosure provides in accordance with yet some aspects, a method for determining the severity of a disease or disorder in a subject, the method comprising administering to said subject a compound, imaging and/or acquiring a spectrum the subject's body or region thereof to obtain at least one imaging parameter, and comparing said at least one imaging parameter to at least one parameter obtained from said subject at the onset of treatment or prior to treatment commencement, wherein the comparison permits determining the severity of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI) or a pharmaceutically acceptable salt, solvate, hydrate, any isomer thereof, any metabolite thereof or physiologically functional derivative thereof, or (f) a combination thereof.

The present disclosure provides in accordance with yet some aspects, a method for determining the effectiveness of a therapeutic treatment of a disease or disorder in a subject, the method comprising administering to said subject a compound, imaging the subject's body or region thereof to obtain at least one imaging parameter, and comparing said at least one imaging parameter to at least one parameter obtained from said subject at the onset of treatment or prior to treatment commencement, wherein the comparison permits determining the effectiveness of the therapeutic treatment of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI) or a pharmaceutically acceptable salt, solvate, hydrate, any isomer thereof, any metabolite thereof or physiologically functional derivative thereof, or (f) a combination thereof.

The present disclosure provides in accordance with yet some aspects, a method of treating, preventing, inhibiting, reducing, eliminating, protecting or delaying the onset of at least one condition in a subject in need thereof, said method comprising the steps of: (a) diagnosis a disease or condition in a subject as described herein and (b) administering to the subject a treatment regimen based on said disease or condition thereby treating the subject.

The present disclosure provides in accordance with yet some aspects, use of at least one 2DG compound in the preparation of a formulation or a composition, wherein the compound is wherein said 2DG compound is deuterium-labeled 2DG.

### EMBODIMENTS

Some embodiments of this disclosure will now be described in the following numbered paragraph. The following description intends to add on the above general description and not limit it in any manner.
1. A 2-deoxy-D-glucose (2DG) compound comprising one or more isotopically substituted hydrogen atoms for use in a method of acquiring at least one magnetic resonance (MR) spectrum and/or at least one MR image.
2. A 2DG compound comprising one or more isotopically substituted hydrogen atoms for use in a method of acquiring at least one MR spectrum and/or at least one MR image from a subject.
3. A 2DG compound comprising one or more isotopically substituted hydrogen atoms for use in a method of diagnosing a subject.
4. A 2DG compound comprising one or more isotopically substituted hydrogen atoms for use in a method of diagnosing a disease in a subject.
5. A pharmaceutical composition comprising at least one 2DG compound, wherein the compound comprises one or more isotopically substituted hydrogen.
6. A diagnostic formulation comprising at least one 2DG compound, wherein the compound comprises one or more isotopically substituted hydrogen.
7. A kit comprising at least one 2DG compound, wherein the compound comprises one or more isotopically substituted hydrogen.
8. A method of acquiring at least one MR spectrum and/or at least one MR image from a subject, the method comprising monitoring a signal from a subject, the subject having been administered at least one 2DG compound to thereby acquire at least one MR image, wherein the 2DG compound comprises one or more isotopically substituted hydrogen.
9. A method of imaging a subject, the method comprising monitoring a signal from a subject using an MR imaging method, the subject having been administered at least one 2DG compound to thereby acquire at least one MR image, wherein the 2DG compound comprising one or more isotopically substituted hydrogen.
10. A method for diagnosis of a disease or condition in a subject, the method comprising: (i) provided a diagnostically effective amount of at least one 2DG compound to the subject and (ii) obtaining an image and/or spectrum of the subject or of a body region of the subject; (iii) identifying body regions in which the compound, or any metabolite thereof has been localized, wherein the 2DG compound comprises one or more isotopically substituted hydrogen; thereby enabling the diagnosis of a disease or condition in a subject according to the localization of the 2DG compound or its metabolites.
11. A method for monitoring a disease state in a subject, the method comprising: (i) provided the subject has been administered a compound; (ii) imaging the subject's body or any one or more regions thereof, in order to obtain at least one imaging parameter indicative of the disease or disorder state; and (iii) comparing the at least one imaging parameter to at least one parameter obtained from the subject at an earlier timepoint; wherein the comparison enables determining the progression of the disease or disorder state, and thereby monitoring the disease state of the subject; and wherein the compound comprises one or more isotopically substituted hydrogen.
12. A method for determining the severity of a disease or disorder in a subject, the method comprising: (i) provided the subject has been administered a compound, (ii) imaging and/or acquiring a spectrum of the subject's body or region thereof to obtain at least one imaging parameter, and (iii) comparing the at least one imaging parameter to at least one parameter obtained from the subject at the onset of treatment or prior to treatment commencement, and thereby determining the severity of the disease or disorder in the subject, wherein the compound comprises one or more isotopically substituted hydrogen.
13. A method for determining the effectiveness of a therapeutic treatment of a disease or disorder in a subject, the method comprising: (i) provided the subject has been administered a compound, (ii) imaging the subject's body or region thereof and thereby obtaining at least one imaging parameter, and (iii) comparing the at least one imaging parameter to at least one parameter obtained from the subject at the onset of treatment or prior to treatment commencement; and thereby determining the effectiveness of the therapeutic treatment of the disease or disorder in the subject, wherein the compound comprises one or more isotopically substituted hydrogen.
14. A method of treating a subject in need thereof, the method comprising the steps of: (i) provided the subject has been administered a compound, (ii) imaging the subject's body or region thereof and thereby obtaining at least one imaging parameter, (iii) obtaining a diagnosis of a disease or condition in a subject according to the localization of the 2DG compound or its metabolites; (iv) administering a treatment to the subject compatible with the diagnosis obtained on step (iii), wherein the compound comprises one or more isotopically substituted hydrogen.
15. A method of treating, preventing, inhibiting, reducing, eliminating, protecting or delaying the onset of at least one condition in a subject in need thereof, said method comprising the steps of: (a) obtaining a diagnosis of a disease or condition in a subject, wherein said diagnosis comprises: (i) provided a diagnostically effective amount of at least one 2DG compound has been administered to the subject; (ii) obtaining an image and/or spectrum of the subject or of a body region of the subject; (iii) identifying body regions in which the compound or any metabolite thereof has been localized, wherein the 2DG compound comprises one or more isotopically substituted hydrogen; and thereby enabling the diagnosis of a disease or condition in a subject according to the localization of the 2DG compound or its metabolites; and (iv) administering to the subject a treatment regimen based on said disease or condition thereby treating, preventing, inhibiting, reducing, eliminating, protecting or delaying the onset of at least one condition in the subject.
16. A method comprising: administering to a subject a 2-deoxy-D-glucose (2DG) compound wherein the 2DG compound is 2-deoxy-D-glucose having one or more carbon-bound hydrogen atoms replaced with deuterium (²H); and acquiring at least one ²H magnetic resonance (MR) spectrum and/or at least one ²H MR image from the subject.
17. A method comprising: administering to a subject a deuterium-labeled 2DG compound wherein one or more deuterium atoms are directly bound to carbon atoms of the 2DG ring; and acquiring at least one ²H MR image using an acquisition sequence configured to preferentially detect signal from said deuterium-labeled 2DG compound relative to naturally occurring HDO by exploiting a difference in longitudinal relaxation time (T1) between said compound and HDO.
18. A method for determining the severity of a disease or disorder in a subject, the method comprising (i) administering to the subject a deuterium-labeled 2DG compound, (ii) acquiring at least one ²H MR imaging parameter from the subject's body or region thereof, (iii) comparing said imaging parameter to at least one parameter obtained from said subject at an earlier timepoint or prior to treatment commencement; thereby determining the severity of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), Formula (XX), Formula (XXI), or a pharmaceutically acceptable salt, solvate, hydrate, any isomer thereof, any metabolite thereof or physiologically functional derivative thereof, or (f) a combination thereof.
19. A method for determining the effectiveness of a therapeutic treatment of a disease or disorder in a subject, the method comprising (i) administering to the subject a deuterium-labeled 2DG compound; (ii) acquiring at least one ²H MR imaging parameter from the subject's body or region thereof, (iii) comparing said imaging parameter to at least one parameter obtained from said subject at an earlier timepoint or prior to treatment commencement; thereby determining the effectiveness of the therapeutic treatment of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), Formula (XX), Formula (XXI), or a pharmaceutically acceptable salt, solvate, hydrate, any isomer thereof, any metabolite thereof or physiologically functional derivative thereof, or (f) a combination thereof.
20. A method comprising: administering to a subject a deuterium-labeled 2DG compound, wherein one or more deuterium atoms are directly bound to carbon atoms of the 2DG molecule; and acquiring at least one ²H MR image from the subject using an acquisition sequence configured to preferentially detect signal from said deuterium-labeled 2DG compound and/or a metabolite thereof relative to naturally occurring deuterium in body water (HDO), wherein said preferential detection exploits a shorter longitudinal relaxation time (T1) of deuterium nuclei bound to carbon atoms of said compound relative to the T1 of deuterium in HDO.
21. Use of at least one DG labeled compound for the preparation of a formulation and/or composition, wherein the compound comprises one or more isotopically substituted hydrogen.
22. The 2DG compound, the pharmaceutical composition, the diagnostic formulation, the kit, the methods, the use of any one of the preceding Embodiments, wherein the 2DG compound is at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof.
23. The 2DG compound, the pharmaceutical composition, the diagnostic formulation, the kit, the methods, the use of any one of the preceding Embodiments, wherein the 2DG compound is [²H₈]2-deoxy-D-glucose.
24. The 2DG compound, the pharmaceutical composition, the diagnostic formulation, the kit, the methods, the use of any one of the preceding Embodiments, wherein the 2DG compound is [²H₂]2-deoxy-D-glucose.
25. The 2DG compound, the pharmaceutical composition, the diagnostic formulation, the kit, the methods, the use of any one of the preceding Embodiments, wherein the compound is represented by Formula (I), Formula (I) or Formula (I′) and/or Formula (II), and/or Formula (II′), and/or Formula (III), and/or Formula (III′), or a pharmaceutically acceptable salt, solvate, hydrate, any tautomer thereof, any isomer thereof, any metabolite thereof or physiologically functional derivative thereof, wherein at least one of X₁, X₂, X₃, X₄, X₅, X₆, X₇, X₈ is ²H (D).
26. The 2DG compound, the pharmaceutical composition, the diagnostic formulation, the kit, the methods, the use of any one of the preceding Embodiments, wherein in the compound is represented by Formula (I), Formula (I) or Formula (I′) and/or Formula (II), and/or Formula (II′), and/or Formula (III), and/or Formula (III′), one or more of (i) one of X₁, X₂, X₃, X₄, X₅, X₆, X₇, and X₈ is ²H, (ii) two of X₁, X₂, X₃, X₄, X₅, X₆, X₇, and X₈ is ²H, (iii) three of X₁, X₂, X₃, X₄, X₅, X₆, X₇, and X₈ is ²H, (iv) four of X₁, X₂, X₃, X₄, X₅, X₆, X₇, and X₈ is ²H, (v) five of X₁, X₂, X₃, X₄, X₅, X₆, X₇, and X₈ is ²H, (vi) six of X₁, X₂, X₃, X₄, X₅, X₆, X₇, and X₈ is ²H, (vii) seven of X₁, X₂, X₃, X₄, X₅, X₆, X₇, and X₈ is ²H, (viii) eight of X₁, X₂, X₃, X₄, X₅, X₆, X₇, and X₈ is ²H or a combination thereof.
27. The 2DG compound, the pharmaceutical composition, the diagnostic formulation, the kit, the methods, the use of any one of the preceding Embodiments, wherein the compound is represented by Formula (IV), Formula (V), Formula (VI), Formula (VII), Formula (VIII), Formula (IX), Formula (X), Formula (XI) or a pharmaceutically acceptable salt, solvate, hydrate, any isomer thereof, any metabolite thereof or physiologically functional derivative thereof.
28. The 2DG compound, the pharmaceutical composition, the diagnostic formulation, the kit, the methods, the use of any one of the preceding Embodiments, wherein the compound is represented by Formula (IV), or Formula (V) or a pharmaceutically acceptable salt, solvate, hydrate, any isomer thereof, any tautomer thereof, any metabolite thereof or physiologically functional derivative thereof.
29. The 2DG compound, the pharmaceutical composition, the diagnostic formulation, the kit, the methods, the use of any one of the preceding Embodiments, wherein the compound is represented by Formula (VI), or Formula (VII) or a pharmaceutically acceptable salt, solvate, hydrate, any isomer thereof, any tautomer thereof, any metabolite thereof or physiologically functional derivative thereof.
30. The 2DG compound, the pharmaceutical composition, the diagnostic formulation, the kit, the methods, the use of any one of the preceding Embodiments, wherein the compound is represented by Formula (VIII), or Formula (IX) or a pharmaceutically acceptable salt, solvate, hydrate, any isomer thereof, any tautomer thereof, any metabolite thereof or physiologically functional derivative thereof.
31. The 2DG compound, the pharmaceutical composition, the diagnostic formulation, the kit, the methods, the use of any one of the preceding Embodiments, wherein the compound is represented by Formula (X), or Formula (XI) or a pharmaceutically acceptable salt, solvate, hydrate, any isomer thereof, any tautomer thereof, any metabolite thereof or physiologically functional derivative thereof.
32. The 2DG compound, the pharmaceutical composition, the diagnostic formulation, the kit, the methods, the use of any one of the preceding Embodiments, wherein the compound has a T₁ relaxation of a ²H nucleus of between about 5 milliseconds to about 200 milliseconds.
33. The 2DG compound, the pharmaceutical composition, the diagnostic formulation, the kit, the methods, the use of any one of the preceding Embodiments, wherein the method comprises acquiring at least one MR spectrum and/or at least one MR image from a subject.
34. The 2DG compound, the pharmaceutical composition, the diagnostic formulation, the kit, the methods, the use of any one of the preceding Embodiments, for use in a method of diagnosing the subject.
35. The 2DG compound, the pharmaceutical composition, the diagnostic formulation, the kit, the methods, the use of any one of the preceding Embodiments, for use in a method of diagnosing a disease in the subject.
36. The 2DG compound, the pharmaceutical composition, the diagnostic formulation, the kit, the methods, the use of any one of the preceding Embodiments, for use in detecting a metabolite of the compound.
37. The 2DG compound, the pharmaceutical composition, the diagnostic formulation, the kit, the methods, the use of any one of the preceding Embodiments, wherein the metabolite is one of [D]2-deoxy-D-glucose-6-phosphate, [D₂]2-deoxy-D-glucose-6-phosphate, [D₃]2-deoxy-D-glucose-6-phosphate, [D₄]2-deoxy-D-glucose-6-phosphate, [D₅]2-deoxy-D-glucose-6-phosphate, [D₆]2-deoxy-D-glucose-6-phosphate, [D₇]2-deoxy-D-glucose-6-phosphate, [D₈]2-deoxy-D-glucose-6-phosphate or a combination thereof.
38. The 2DG compound, the pharmaceutical composition, the diagnostic formulation, the kit, the methods, the use of any one of the preceding Embodiments, wherein the metabolite is a phosphorylation product of a compound represented by one or more of Formula (I), Formula (II), Formula (III), Formula (IV), Formula (V), Formula (VI), Formula (VII), Formula (VIII), Formula (IX), Formula (X), Formula (XI) or any combination thereof.
39. The 2DG compound, the pharmaceutical composition, the diagnostic formulation, the kit, the methods, the use of any one of the preceding Embodiments, wherein the metabolite is represented by one or more of Formula (XII), Formula (XIII), Formula (XIV), Formula (XV), Formula (XVI), Formula (XVII), Formula (XVIII) or any combination thereof.
40. The 2DG compound, the pharmaceutical composition, the diagnostic formulation, the kit, the methods, the use of any one of the preceding Embodiments, for use in (i) a method of monitoring a disease state in the subject and/or (ii) a method of determining a site of a disease in the subject.
41. The 2DG compound, the pharmaceutical composition, the diagnostic formulation, the kit, the methods, the use of any one of the preceding Embodiments, wherein the disease is associated with increased glucose metabolism.
42. The 2DG compound, the pharmaceutical composition, the diagnostic formulation, the kit, the methods, the use of any one of the preceding Embodiments, wherein the disease may be oncological, neurological, psychiatric, cardiovascular, infectious or inflammatory.
43. The 2DG compound, the pharmaceutical composition, the diagnostic formulation, the kit, the methods, the use of any one of the preceding Embodiments, wherein the disease is a proliferative disorder.
44. The 2DG compound, the pharmaceutical composition, the diagnostic formulation, the kit, the methods, the use of any one of the preceding Embodiments, wherein the disease is ischemia.
45. The 2DG compound, the pharmaceutical composition, the diagnostic formulation, the kit, the methods, the use of any one of the preceding Embodiments, for distinguishing between healthy and abnormal tissues or organs and/or distinguishing or differentiating between malignant and benign tumors.
46. The 2DG compound, the pharmaceutical composition, the diagnostic formulation, the kit, the methods, the use of any one of the preceding Embodiments, wherein at least one MR image is at least one ²H image.
47. The 2DG compound, the pharmaceutical composition, the diagnostic formulation, the kit, the methods, the use of any one of the preceding Embodiments, wherein the at least one ²H image is acquired at an acquisition time of at most about 5 minutes.
48. The 2DG compound, the pharmaceutical composition, the diagnostic formulation, the kit, the methods, the use of any one of the preceding Embodiments, wherein the at least one ²H MR image from a subject with a spatial resolution of between about 2 mm and about 20 mm, at times between about 2 mm and about 10 mm, at times between about 2 mm and about 5 mm (in plane resolution).
49. The 2DG compound, the pharmaceutical composition, the diagnostic formulation, the kit, the methods, the use of any one of the preceding Embodiments, wherein at least one MR image is of at least one compound or a metabolite thereof.
50. The 2DG compound, the pharmaceutical composition, the diagnostic formulation, the kit, the methods, the use of any one of the preceding Embodiments, for determining a site of a disease and/or for distinguishing between healthy and abnormal tissues or organs.
51. The 2DG compound, the pharmaceutical composition, the diagnostic formulation, the kit, the methods, the use of any one of the preceding Embodiments, for distinguishing or differentiating between malignant and benign tumors.
52. The 2DG compound, the pharmaceutical composition, the diagnostic formulation, the kit, the methods, the use of any one of the preceding Embodiments, wherein the method comprises prior to the administration step, acquiring at least one ²H image from the subject's body or one or more body regions.
53. The 2DG compound, the pharmaceutical composition, the diagnostic formulation, the kit, the methods, the use of any one of the preceding Embodiments, wherein the method comprises comparing at least one parameter obtained from the at least one ²H MR image to at least one parameter obtained from the at least one ²H MR image in the same subject at an earlier timepoint, wherein the comparison enables diagnosis of the disease.
54. The 2DG compound, the pharmaceutical composition, the diagnostic formulation, the kit, the methods, the use of any one of the preceding Embodiments, wherein the method comprises administering to a subject diagnosed with a disease, a treatment regimen based on said disease or condition thereby treating, preventing, inhibiting, reducing, eliminating, protecting or delaying the onset of the disease.
55. The 2DG compound, the pharmaceutical composition, the diagnostic formulation, the kit, the methods, the use of any one of the preceding Embodiments, wherein the treatment regimen is designed for treating, preventing, inhibiting, reducing, eliminating, protecting or delaying a disease is associated with increased glucose metabolism.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1 are deuterium Nuclear Magnetic Resonance (NMR) spectra of brain slices perfused with artificial cerebrospinal fluid (aCSF) containing [6,6-D₂]D-glucose with two ischemic periods, D-Glc, [6,6-D₂]D-glucose; D-Lac, [3,3-D₂]lactate.

FIGS. 2A-2C show individual time courses of [6,6-D₂]D-glucose (D-Glc) and [3,3-D₂]lactate (D-Lac) in brain slices on three experimental days, respectively, Y-axis shows concentration in the NMR tube in mM for the three species (HDO, D-Glc, and D-Lac), the breaks in D-NMR acquisitions in FIG. 2C were taken to record ³¹P spectra.

FIGS. 3A-3H are ³¹P NMR spectra of the brain slices during the three ischemia experiments; FIG. 3A is a ³¹P NMR spectrum at day 1 before 1^{st} ischemia; FIG. 3B is a ³¹P NMR spectrum at day 1 after 2^{nd} ischemia; FIG. 3C is a ³¹P NMR spectrum at day 2 before 1^{st} ischemia; FIG. 3D is a ³¹P NMR spectrum at day 2 after 2^{nd} ischemia; FIG. 3E is a ³¹P NMR spectrum at day 3 before 1^{st} ischemia; FIG. 3F is a ³¹P NMR spectrum at day 3 after 2^{nd} ischemia; FIG. 3G is a ³¹P NMR spectrum at day 3 during 3^{rd} ischemia; FIG. 3H is a ³¹P NMR spectrum at day 3 after 3^{rd} ischemia.

FIG. 4 are stacked deuterium NMR spectra of an experiment with three ischemic conditions, only the third ischemic duration is shown, D-Glc, [6,6-D₂]D-glucose; D-Lac, [3,3-D₂]lactate; D-Glx deuterium-labeled-glutamate and -glutamine FIG. 5 shows partial time course of the experiment with three ischemic conditions, for clarity, the D-Glx level was multiplied 5-fold compared to HDO, D-Glc, and D-Lac, the X-axis represents 2-minute intervals, and X=0 is at the start of the reperfusion after the first ischemic condition, where the D-Glx signal was first observed.

FIGS. 6A and 6B are ¹H NMR spectra of commercial 2-deoxyglucose-6,6-H₂ (FIG. 6A) and 2-deoxyglucose-6,6-D₂ (FIG. 6B).

FIG. 7 is a ²H-NMR spectrum of a water-soluble extract from a rat brain injected with [²H₄]2-deoxy-D-glucose ([3,4,6,6-D4]2-deoxy-D-glucose), the right signal (full arrow) is of deuterons of [3,4,6,6-D4]2-deoxy-D-glucose and the left signal (dashed arrow) is of natural abundance of deuterium in water (HDO) and was truncated for better visibility, rat was awake for 30 min after administration and then sacrificed by CO₂ overdose and the brain was immediately excised and freeze-clamped, the water-soluble metabolites were extracted using a chloroform:methanol:water 1:1:1 extraction procedure.

### DETAILED DESCRIPTION OF EMBODIMENTS

The diagnosis of disease, particularly early diagnosis, is critically important as it assists in identifying disease, tracking its progression, determining appropriate treatment regimens and planning follow-ups measures.

Imaging and spectroscopic techniques, such as positron emission tomography (PET), computed tomography (CT), magnetic resonance spectroscopy (MRS) and magnetic resonance imaging (MRI), are commonly employed for diagnosis a wide range of disease and condition. Among others, PET and CT involve the use of ionizing radiation, whereas MRS and MRI are non-invasive, safe non-radioactive diagnostic methods.

Disease diagnosis using PET frequently relies on the contrast agent ¹⁸F-fluoro-2-deoxy-D-glucose (FDG), a fluorinated analogue of 2-deoxy-D-glucose (2DG). 2DG is a non-metabolic derivative of D-glucose that enters cells via the glucose transporters and subsequently phosphorylated by hexokinases into a phosphorylated product. This product (may also be considered as a metabolite) is largely non-metabolized and does not proceed through the glycolysis pathway, resulting in its accumulation within the cells. This accumulated compound (metabolite) can be visualized following administration, enabling the diagnosis of tissues exhibiting high glucose uptake and phosphorylation.

However, the use of ionising radiation associated with the FDG-PET poses significant limitations, including restrictions on the frequency of examinations, prolonged intervals between repeated scans and concerns regarding its use in vulnerable populations such as children and pregnant women.

The present disclosure relates to 2-deoxy-D-glucose (2DG) compounds, in which one or more carbon-bound hydrogen atoms are replaced with deuterium (²H or D), a stable, non-radioactive hydrogen isotope, that may be used as contrast agents in non-invasive MR methods.

The deuterium-labeled 2DG compounds described herein provide a magnetic resonance spectroscopy (MRS) and/or magnetic resonance imaging (MRI) advantage by virtue of relaxation properties of one or more deuterons of the administered compound and/or one or more metabolites thereof. It was suggested that the one or more such deuterons exhibit a longitudinal relaxation time (T1) shorter than that of naturally occurring deuterium-containing species in the subject, including deuterium in water (HDO). This difference may be exploited in a ²H magnetic resonance acquisition to preferentially detect the administered compound and/or one or more metabolites thereof relative to HDO, thereby reducing background signal contribution, improving contrast or detectability, and/or permitting faster image acquisition.

Hence, in accordance with some aspects, the present disclosure provides a 2DG compound comprising one or more isotopically substituted hydrogen atoms for use in a method of acquiring at least one MR spectrum and/or at least one MR image.

In the following text, when referring to the 2DG compound or compound it is to be understood as also referring to pharmaceutical compositions, methods, kits and uses, disclosed herein. Thus, whenever providing a feature with reference to the 2DG compound or compounds, it is to be understood as defining the same feature with respect to the pharmaceutical compositions, the methods, the kits and the uses, mutatis mutandis.

2-Deoxyglucose is also known by the IUPAC name 2-Deoxy-D-arabino-hexopyranose, by the systematic IUPAC name (4R,5S,6R)-6-(hydroxymethyl)oxane-2,4,5-triol as well as by other names: 2-Deoxy-d-mannose, 2-Deoxy-d-arabino-hexose or 2-DG. It has CAS Number of 154-17-6.

As appreciated, 2DG may exist in various forms, such as open-chain and cyclic (pyranose and furanose) structures, which can interconvert depending on the chemical environment and specific conditions.

In some embodiments, the deuterium-labeled 2DG compound comprises one or more deuterons bound to carbon atoms in a cyclic form of the compound, and such deuterons exhibit relaxation behavior different from that of deuterium present in HDO and/or deuterium present in compound or other compounds in the subject's body. In some embodiments, the acquisition is configured to exploit such differences in relaxation behavior to improve detection, visualization, localization, or quantification of the administered deuterium-labeled 2DG compound and/or one or more metabolites thereof.

The 2DG compound as used herein may refer to the linear form and to the cyclic (ring) form as is further shown herein below.

In accordance with the present disclosure, reference to 2DG compound encompasses any tautomer thereof or isomer thereof.

The 2DG compound as described herein comprises one or more isotopically substituted hydrogen atom is referred herein as a “labeled compound,” “isotopically enriched 2DG compound” or “isotopologue 2DG compound”.

In some examples, isotopologue 2DG compound may be employed in the diagnosis and prognosis of pathological conditions.

In accordance with some other aspects, it is provided a 2DG compound comprising one or more isotopically substituted hydrogen atoms for use in a method of diagnosing a condition or disease in a subject.

The term “isotopically substituted hydrogen atom” or “isotopically labeled hydrogen atom” is meant to encompasses a hydrogen atom in the 2DG compound that has been replaced/substituted with a different isotope of hydrogen. The different isotope of hydrogen has an atomic mass which is different than the atomic mass of the prevalent naturally abundant isotope of the same atom. Due to different number of neutrons in the nuclei, the atomic mass of an isotopically labeled atoms is different. The total number of neutrons and protons in the nucleus represents its isotopic number.

In the context of this disclosure, the term “isotope labeling” (or “isotopically substituted atom”) explicitly excludes radioactive labeling. In other words, the present disclosure pertains solely to stable isotopes and excludes the use of radioactive isotopes in the described compounds.

Natural hydrogen has two stable (non-radioactive) isotopes, hydrogen-1 (H-1 or H or hydrogen), which represents the majority of naturally occurring hydrogen, and hydrogen-2 (H-2 or ²H or D or deuterium), which is less common. Each of H-1 or D has 1 proton, hydrogen has no neutrons and deuterium has 1 neutron.

The isotopically labeled hydrogen or isotopically substituted hydrogen atom refers in some examples to ²H (D, deuterium).

In some examples, the 2DG compound comprises one or more deuterium atoms.

When referring to a compound comprising at least one isotopically substituted atom (i.e. the isotopologue 2DG compound), it should be understood to include compounds in which the isotopically substituted atoms are present at levels above their natural abundance. In other words, the isotopologue 2DG compound is isotopically enriched with deuterium (²H).

In embodiments, the isotopic enrichment of the compound is between about 0.015% and about 99.9%. Thus, a compound as described herein may exhibit varying degrees of isotopic enrichment.

Unless otherwise stated, the terms compound, labeled compound or isotopically substituted compound, refers to a 2DG compound in which one or more hydrogen atom(s) was replaced (also denoted herein as substituted or labelled) with one or more (at least one) deuterium (²H) atom.

It should be noted that the present disclosure also encompasses such 2DG compounds, which are optionally, isotopically substituted with one or more additional atom, such as nitrogen or carbon atoms.

In some embodiments, the 2DG compound of the invention comprises at least one substituted hydrogen atom, at times at least two substituted hydrogen atoms, at times at least three substituted hydrogen atoms, at times at least four substituted hydrogen atoms, at times at least five substituted hydrogen atoms, at times at least six substituted hydrogen atoms, at times even at least seven substituted hydrogen atoms. As noted the hydrogen atom(s) is substituted by deuterium atom.

In some embodiments, the 2-deoxy-D-glucose compound comprises one ²H atom. In some embodiments, the compound is [²H]2-deoxy-D-glucose. In some embodiments, the 2-deoxy-D-glucose compound comprises two ²H atoms. In some embodiments, the compound is [²H₂]2-deoxy-D-glucose. In some embodiments, the 2-deoxy-D-glucose compound comprises three ²H atoms. In some embodiments, the compound is [²H₃]2-deoxy-D-glucose. In some embodiments, the 2-deoxy-D-glucose compound comprises four ²H atoms. In some embodiments, the compound is [²H₄]2-deoxy-D-glucose. In some embodiments, the 2-deoxy-D-glucose compound comprises five ²H atoms. In some embodiments, the compound is [²H₅]2-deoxy-D-glucose. In some embodiments, the 2-deoxy-D-glucose compound comprises six ²H atoms. In some embodiments, the compound is [²H₆]2-deoxy-D-glucose. In some embodiments, the 2-deoxy-D-glucose compound comprises seven ²H atoms. In some embodiments, the compound is [²H₇]2-deoxy-D-glucose. In some embodiments, the 2-deoxy-D-glucose compound comprises eight ²H atoms. In some embodiments, the compound is [²H₈]2-deoxy-D-glucose

In some embodiments, the compound is or comprises at least one of [²H]2-deoxy-D-glucose (one deuterium atom), [²H₂]2-deoxy-D-glucose (two deuterium atoms), [²H₃]2-deoxy-D-glucose (three deuterium atoms), [²H₄]2-deoxy-D-glucose (four deuterium atoms), [²H₅]2-deoxy-D-glucose (five deuterium atoms), [²H₆]2-deoxy-D-glucose (six deuterium atoms), [²H₇]2-deoxy-D-glucose (seven deuterium atoms), [²H₈]2-deoxy-D-glucose (eight deuterium atoms) or a combination thereof.

As is well understood, the isotopic substitution of specific atoms in a compound described herein can be achieved using techniques known to those skilled in the art, such as synthesizing the compound from isotopically enriched reactants or by directly enriching specific nuclei of the compound with isotopes.

These atom substitutions render the 2DG compounds particularly useful for MR applications.

Magnetic resonance (MR) methods, unlike positron emission tomography (PET), are not associated with ionizing radiation and do not require the use of a radioactive contrast agent such as FDG. Consequently, the application of a non-radioactive, deuterium-labeled 2DG in MR methods presents a safer alternative to FDG-PET examinations.

Hence, in accordance with some aspects, the present disclosure provides a compound comprises at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof for use in a method of acquiring at least one magnetic resonance (MR) spectrum and/or at least one MR image.

As described herein, the isotopologue compound is proposed as a promising candidate for the diagnosis of various diseases and conditions.

In some embodiments which may be considered as aspects of the present disclosure, the compound comprising at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof is for use in a method of diagnosing a condition or disease in a subject.

In some examples, the compound is or comprises [²H]2-deoxy-D-glucose.

In accordance with some aspects, the present disclosure provides [²H]2-deoxy-D-glucose for use in a method of acquiring at least one MR spectrum and/or at least one MR image. In some embodiments which may be considered as aspects of the present disclosure, the compound [D]2-deoxy-D-glucose is for use in a method of diagnosing a condition or disease in a subject.

In some examples, the compound is or comprises [²H₂]2-deoxy-D-glucose.

Hence, in accordance with some aspects, the present disclosure provides [²H₂]2-deoxy-D-glucose for use in a method of acquiring at least one MR spectrum and/or at least one MR image. In some embodiments which may be considered as aspects of the present disclosure, the compound [D₂]2-deoxy-D-glucose is for use in a method of diagnosing a condition or disease in a subject.

In some examples, the compound is or comprises [²H₃]2-deoxy-D-glucose.

Hence, in accordance with some aspects, the present disclosure provides [²H₃]2-deoxy-D-glucose for use in a method of acquiring at least one MR spectrum and/or at least one MR image. In some embodiments which may be considered as aspects of the present disclosure, the compound [D₃]2-deoxy-D-glucose is for use in a method of diagnosing a condition or disease in a subject.

In some examples, the compound is or comprises [²H₄]2-deoxy-D-glucose.

Hence, in accordance with some aspects, the present disclosure provides [²H₄]2-deoxy-D-glucose for use in a method of acquiring at least one MR spectrum and/or at least one MR image. In some embodiments which may be considered as aspects of the present disclosure, the compound [D₄]2-deoxy-D-glucose is for use in a method of diagnosing a condition or disease in a subject.

In some examples, the compound is or comprises [²H₅]2-deoxy-D-glucose.

Hence, in accordance with some aspects, the present disclosure provides [²H₅]2-deoxy-D-glucose for use in a method of acquiring at least one MR spectrum and/or at least one MR image. In some embodiments which may be considered as aspects of the present disclosure, the compound [D₅]2-deoxy-D-glucose is for use in a method of diagnosing a condition or disease in a subject.

In some examples, the compound is or comprises [²H₆]2-deoxy-D-glucose.

Hence, in accordance with some aspects, the present disclosure provides [²H₆]2-deoxy-D-glucose for use in a method of acquiring at least one MR spectrum and/or at least one MR image. In some embodiments which may be considered as aspects of the present disclosure, the compound [D₆]2-deoxy-D-glucose is for use in a method of diagnosing a condition or disease in a subject.

In some examples, the compound is or comprises [²H₇]2-deoxy-D-glucose.

Hence, in accordance with some aspects, the present disclosure provides [²H₇]2-deoxy-D-glucose for use in a method of acquiring at least one MR spectrum and/or at least one MR image. In some embodiments which may be considered as aspects of the present disclosure, the compound [D₇]2-deoxy-D-glucose is for use in a method of diagnosing a condition or disease in a subject.

In some examples, the compound is or comprises [²H₈]2-deoxy-D-glucose.

Hence, in accordance with some aspects, the present disclosure provides [²H₈]2-deoxy-D-glucose for use in a method of acquiring at least one MR spectrum and/or at least one MR image. In some embodiments which may be considered as aspects of the present disclosure, the compound [D₈]2-deoxy-D-glucose is for use in a method of diagnosing a condition or disease in a subject.

The position at which the one or more hydrogen atoms is substituted is not limited to a specific position.

In accordance with some aspects, the present disclosure provides a compound for use in a method of acquiring at least one MR spectrum and/or at least one MR image, wherein the compound is a represented by Formula (I):

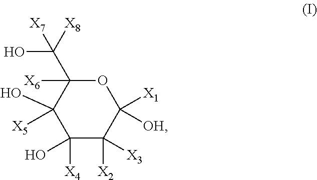

or a pharmaceutically acceptable salt, solvate, hydrate, any tautomer thereof, any isomer thereof, any metabolite thereof or physiologically functional derivative thereof, wherein at least one of X₁, X₂, X₃, X₄, X₅, X₆, X₇, X₈ is ²H (D).

In accordance with some aspects, the present disclosure provides a compound for use in a method of diagnosing a disease in a subject, wherein said compound is represented by Formula (I) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

As described herein, the compounds represented by a cyclic form as in the compound represented by Formula (I) encompass the cyclic form as well as the linear form represented below by Formula (I′).

As appreciated, both forms may be in equilibrium under appropriate conditions. In addition, the compound represented by Formula (I) and/or Formula (I′) encompasses any isomer thereof.

In accordance with some aspects, the present disclosure provides a compound for use in a method of acquiring at least one MR spectrum and/or at least one MR image, wherein the compound is a represented by Formula (I′):

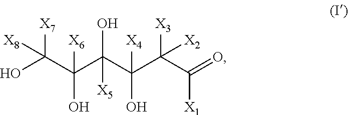

or a pharmaceutically acceptable salt, solvate, hydrate, any tautomer thereof, any isomer thereof, any metabolite thereof or physiologically functional derivative thereof, wherein at least one of X₁, X₂, X₃, X₄, X₅, X₆, X₇, X₈ is ²H (D).

In accordance with some aspects, the present disclosure provides a compound for use in a method of diagnosing a disease in a subject, wherein said compound is represented by Formula (I′) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

The compound represented by Formula (I) and/or (I′) encompasses any isomer thereof.

As also described herein below, the term isomer thereof as used herein includes stereoisomers (e.g., D- and L-glucose) and/or anomers (e.g., α- and β-forms).

In some examples, the compound represented by Formula (I) and/or (I′) comprises L-glucose.

In some examples, the compound represented by Formula (I) and/or (I′) comprises D-glucose.

In accordance with some embodiments that may be considered as aspects of the present disclosure, the compound is represented by Formula (II). In accordance with some embodiments that may be considered as aspects of the present disclosure, the compound is represented by Formula (II′).

In accordance with some aspects, the present disclosure provides a compound for use in a method of acquiring at least one MR spectrum and/or at least one MR image, wherein the compound is a represented by Formula (II):

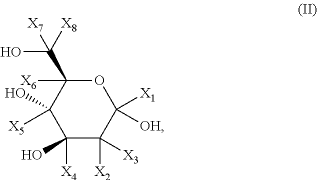

or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some aspects, the present disclosure provides a compound for use in a method of diagnosing a disease in a subject, wherein said compound is represented by Formula (II) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some aspects, the present disclosure provides a compound for use in a method of acquiring at least one MR spectrum and/or at least one MR image, wherein the compound is a represented by Formula (II′):

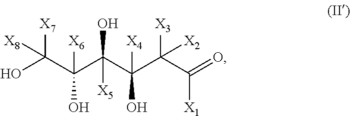

or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some aspects, the present disclosure provides a compound for use in a method of diagnosing a disease in a subject, wherein said compound is represented by Formula (II′) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some embodiments that may be considered as aspects of the present disclosure, the compound is represented by Formula (III):

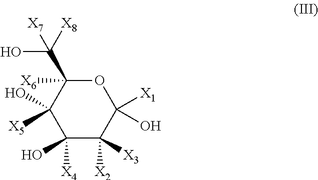

or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

Hence, in accordance with some aspects, the present disclosure provides a compound for use in a method of acquiring at least one MR spectrum and/or at least one MR image, wherein the compound is a represented by Formula (III) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some other aspects, the present disclosure provides a compound for use in a method of diagnosing a disease in a subject, wherein said compound is represented by Formula (III) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

As described, the present disclosure encompasses both the keto and enol forms of the compounds.

The compound described herein may refer in some examples to a mixture of tautomer's represented for example Formula (III) and Formula (III′) as shown below:

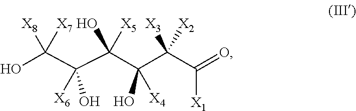

or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

Hence, in accordance with some aspects, the present disclosure provides a compound for use in a method of acquiring at least one MR spectrum and/or at least one MR image, wherein the compound is a represented by Formula (III′) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some other aspects, the present disclosure provides a compound for use in a method of diagnosing a disease in a subject, wherein said compound is represented by Formula (III′) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), one of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), two of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), three of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), four of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), five of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), six of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), seven of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), eight of X₁, X₂, X₃, X₄, X₅, X₆, X₇, X₈ is ²H (D).

In accordance with some embodiments that may be considered as aspects of the present disclosure, in the compound represented by Formula (I) or Formula (I′) and/or Formula (II), and/or Formula (II′), and/or Formula (III), and/or Formula (III′), one or more of the following (i) one of X₁, X₂, X₃, X₄, X₅, X₆, X₇, and X₈ is ²H, (ii) two of X₁, X₂, X₃, X₄, X₅, X₆, X₇, and X₈ is ²H, (iii) three of X₁, X₂, X₃, X₄, X₅, X₆, X₇, and X₈ is ²H, (iv) four of X₁, X₂, X₃, X₄, X₅, X₆, X₇, and X₈ is ²H, (v) five of X₁, X₂, X₃, X₄, X₅, X₆, X₇, and X₈ is ²H, (vi) six of X₁, X₂, X₃, X₄, X₅, X₆, X₇, and X₈ is ²H, (vii) seven of X₁, X₂, X₃, X₄, X₅, X₆, X₇, and X₈ is ²H, (viii) eight of X₁, X₂, X₃, X₄, X₅, X₆, X₇, and X₈ is ²H or a combination thereof.

In some embodiments, in the compound represented by Formula (I), Formula (I′), Formula (II), Formula (II′), Formula (III), and/or Formula (III′), X₇ and X₈ are ²H. In some embodiments, the compound represented by Formula (I), Formula (I′), Formula (II), Formula (II′), Formula (III), and/or Formula (III′), wherein X₇ and X₈ are ²H, is [6,6-D₂]2-deoxy-D-glucose. In some embodiments, the compound represented by Formula (I), Formula (I′), Formula (II), Formula (II′), Formula (III), and/or Formula (III′), wherein X₇ and X₈ are ²H, is [6,6-D₂]2-deoxy-D-glucose, a [²H₂]2-deoxy-D-glucose isotopologue.

In some embodiments, in the compound represented by Formula (I), Formula (I′), Formula (II), Formula (II′), Formula (III), and/or Formula (III′), X₂ and X₃ are ²H. In some embodiments, the compound represented by Formula (I), Formula (I′), Formula (II), Formula (II′), Formula (III), and/or Formula (III′), wherein X₂ and X₃ are ²H, is [2,2-D₂]2-deoxy-D-glucose. In some embodiments, the compound represented by Formula (I), Formula (I′), Formula (II), Formula (II′), Formula (III), and/or Formula (III′), wherein X₂ and X₃ are ²H, is [2,2-D₂]2-deoxy-D-glucose, a [²H₂]2-deoxy-D-glucose isotopologue.

In some embodiments, in the compound represented by Formula (I), Formula (I′), Formula (II), Formula (II′), Formula (III), and/or Formula (III′) X₄, X₅, X₇, and X₈ are ²H. In some embodiments, the compound represented by Formula (I), Formula (I′), Formula (II), Formula (II′), Formula (III), and/or Formula (III′), wherein X₄, X₅, X₇, and X₈ are ²H, is [3,4,6,6-D₄]2-deoxy-D-glucose. In some embodiments, the compound represented by Formula (I), Formula (I′), Formula (II), Formula (II′), Formula (III), and/or Formula (III′), wherein X₄, X₅, X₇, and X₈ are ²H, is [3,4,6,6-D₄]2-deoxy-D-glucose, a [²H₄]2-deoxy-D-glucose isotopologue.

In some embodiments, in the compound represented by Formula (I), Formula (I′), Formula (II), Formula (II′), Formula (III), and/or Formula (III′), wherein one of X₂ and X₃ is ²H, and wherein X₄, X₅, X₇, and X₈ are ²H. In some embodiments, the compound represented by Formula (I), Formula (I′), Formula (II), Formula (II′), Formula (III), and/or Formula (III′), wherein one of X₂ and X₃ is ²H, and wherein X₄, X₅, X₇, and X₈ are ²H, is [2,3,4,6,6-D₅]2-deoxy-D-glucose. In some embodiments, the compound represented by Formula (I), Formula (I′), Formula (II), Formula (II′), Formula (III), and/or Formula (III′), wherein one of X₂ and X₃ is ²H, and wherein X₄, X₅, X₇, and X₈ are ²H, is [2,3,4,6,6-D₅]2-deoxy-D-glucose, a [²H₅]2-deoxy-D-glucose isotopologue.

In some embodiments, in the compound represented by Formula (I), Formula (I′), Formula (II), Formula (II′), Formula (III), and/or Formula (III′) X₁, one of X₂ and X₃, and X₄, X₅, X₇, and X₈ are ²H. In some embodiments, the compound represented by Formula (I), Formula (I′), Formula (II), Formula (II′), Formula (III), and/or Formula (III′), wherein X₁, one of X₂ and X₃, and X₄, X₅, X₇, and X₈ are ²H, is [1,2,3,4,6,6-D₆]2-deoxy-D-glucose. In some embodiments, the compound represented by Formula (I), Formula (I′), Formula (II), Formula (II′), Formula (III), and/or Formula (III′), wherein X₁, one of X₂ and X₃, and X₄, X₅, X₇, and X₈ are ²H, is [1,2,3,4,6,6-D₆]2-deoxy-D-glucose, a [²H₆]2-deoxy-D-glucose isotopologue.

In some embodiments, in the compound represented by Formula (I), Formula (I′), Formula (II), Formula (II′), Formula (III), and/or Formula (III′) X₁, one of X₂ and X₃, and X₄, X₅, X₆, X₇, and X₈ are ²H. In some embodiments, the compound represented by Formula (I), Formula (I′), Formula (II), Formula (II′), Formula (III), and/or Formula (III′), wherein X₁, one of X₂ and X₃, and X₄, X₅, X₆, X₇, and X₈ are ²H, is [1,2,3,4,5,6,6-D₇]2-deoxy-D-glucose. In some embodiments, the compound represented by Formula (I), Formula (I′), Formula (II), Formula (II′), Formula (III), and/or Formula (III′), wherein X₁, one of X₂ and X₃, and X₄, X₅, X₆, X₇, and X₈ are ²H, is [1,2,3,4,5,6,6-D₇]2-deoxy-D-glucose, a [²H₇]2-deoxy-D-glucose isotopologue.

In some embodiments, in the compound represented by Formula (I), Formula (I′), Formula (II), Formula (II′), Formula (III), and/or Formula (III′) is, each of X₁, X₂, X₃, X₄, X₅, X₆, X₇, and X₈ is ²H. In some embodiments, the compound represented by Formula (I), Formula (I′), Formula (II), Formula (II′), Formula (III), and/or Formula (III′), wherein each of X₁, X₂, X₃, X₄, X₅, X₆, X₇, and X₈ is ²H, is [1,2,2,3,4,5,6,6-D₈]2-deoxy-D-glucose. In some embodiments, the compound represented by Formula (I), Formula (I′), Formula (II), Formula (II′), Formula (III), and/or Formula (III′), wherein each of X₁, X₂, X₃, X₄, X₅, X₆, X₇, and X₈ is ²H, is [1,2,2,3,4,5,6,6-D₈]2-deoxy-D-glucose, a [²H₅]2-deoxy-D-glucose isotopologue.

For the sake of simplicity, the compounds below are represented by the closed ring/cyclic form. However, it should be noted that all the compounds provided herein below by the closed ring form also encompass the respective linear form as described herein and exemplified above for compound having Formula (I′) and/or (II′) and/or (III′).

In accordance with some embodiments that may be considered as aspects of the present disclosure, the compound is represented by Formula (IV):

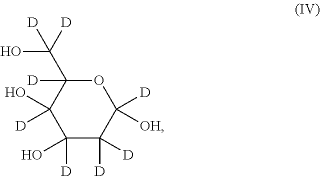 [SMILES: [2H]C([2H])(O)C1([2H])OC([2H])(O)C([2H])([2H])C([2H])(O)C1([2H])O]

or a pharmaceutically acceptable salt, solvate, hydrate, any isomer thereof, any metabolite thereof or physiologically functional derivative thereof.

In some examples, the compound is or comprises [²H₈]2-deoxy-D-glucose wherein the deuterium atoms are located at positions 1, 2, 2, 3, 4, 5, 6, and 6 of the 2-deoxy-D-glucose molecule (i.e., [1,2,2,3,4,5,6,6-D8]2-deoxy-D-glucose).

Hence, in accordance with some aspects, the present disclosure provides a compound for use in a method of acquiring at least one magnetic resonance (MR) spectrum and/or at least one MR image, wherein the compound is a represented by Formula (IV) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (IV) or a pharmaceutically acceptable salt, solvate, hydrate, any isomer thereof, any metabolite thereof or physiologically functional derivative thereof.

It should be noted that in accordance with the present disclosure, a compound represented by Formula (IV) encompasses any isomer thereof.

In some examples, the compound is represented by Formula (V):

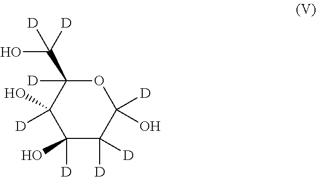 [SMILES: [2H]C1(O)O[C@]([2H])(C([2H])([2H])O)[C@@]([2H])(O)[C@]([2H])(O)C1([2H])[2H]]

or a pharmaceutically acceptable salt, solvate, hydrate, any tautomer thereof, any isomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (V) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (V) or a pharmaceutically acceptable salt, solvate, hydrate, any isomer thereof, any metabolite thereof or physiologically functional derivative thereof.

In some embodiments, the compound represented by Formula (IV) and (V) are denoted as [1,2,2,3,4,5,6,6-D8]2-deoxy-D-glucose.

In some embodiments, the compounds represented by Formulae (IV) and (V) are anomeric forms of [1,2,2,3,4,5,6,6-D8]2-deoxy-D-glucose, also referred to herein as [²H₈]2-deoxy-D-glucose.

As used herein, “[²H₅]2-deoxy-D-glucose” refers to [1,2,2,3,4,5,6,6-D8]2-deoxy-D-glucose and includes the α-anomer, the β-anomer, mixtures thereof, solvates, hydrates, salts, and isotopically equivalent forms thereof, unless the context clearly indicates otherwise.

In accordance with some embodiments that may be considered as aspects of the present disclosure, the compound is a compound represented by Formula (VI):

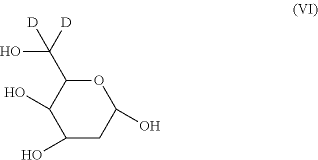 [SMILES: [2H]C([2H])(O)C1OC(O)CC(O)C1O]

or a pharmaceutically acceptable salt, solvate, hydrate, any isomer thereof, any tautomer thereof, any metabolite thereof or physiologically functional derivative thereof.

In some examples, the compound is or comprises [6,6-D₂]2-deoxy-D-glucose, which may also be referred to as [²H₂]2-deoxy-D-glucose.

Hence, in accordance with some aspects, the present disclosure provides a compound for use in a method of acquiring at least one magnetic resonance (MR) spectrum and/or at least one MR image, wherein the compound is a represented by Formula (VI) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (VI) or a pharmaceutically acceptable salt, solvate, hydrate, any isomer thereof, any metabolite thereof or physiologically functional derivative thereof.

It should be noted that in accordance with the present disclosure, a compound represented by Formula (VI) encompasses any isomer thereof. The isomer includes tautomer of compounds represented by Formula (VII).

In some examples, the compound is represented by Formula (VII):

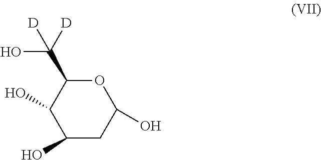 [SMILES: [2H]C([2H])(O)[C@H]1OC(O)C[C@@H](O)[C@@H]1O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (VII) or a pharmaceutically acceptable salt, solvate, hydrate, any isomer thereof, any metabolite thereof or physiologically functional derivative thereof.

In some embodiments, the compound represented by Formula (VI) and (VII) are anomeric forms of [6,6-D2]2-deoxy-D-glucose also denoted as 2-deoxyglucose-6,6-D2.

In some examples, the compound is 2-deoxyglucose-6,6-D2.

In some embodiments, the compounds represented by Formulae (VI) and (VII) are anomeric forms of [6,6-D₂]2-deoxy-D-glucose.

In some embodiments, the compounds represented by Formulae (VI) and (VII) are anomeric forms of [6,6-D₂]2-deoxy-D-glucose, including the α-anomer, the β-anomer, mixtures thereof, solvates, hydrates, and salts thereof.

In accordance with some other aspects, the present disclosure provides a compound for use in a method of diagnosing a subject, wherein said compound is represented by Formula (VII) or a pharmaceutically acceptable salt, solvate, hydrate, any isomer thereof, any metabolite thereof or physiologically functional derivative thereof.

In some examples, the compound is represented by Formula (VII′):

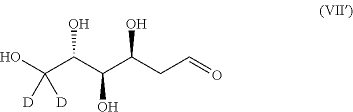 [SMILES: [2H]C([2H])(O)[C@H](O)[C@H](O)[C@@H](O)CC=O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (VII′) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (VII′) or a pharmaceutically acceptable salt, solvate, hydrate, any isomer thereof, any metabolite thereof or physiologically functional derivative thereof.

In some examples, the compound is D-Arabino-2-deoxyhexose-d1.

In some examples, the compound is characterized by a CAS No.: 188004-07-1.

In some examples, the compound is represented by Formula (VIII):

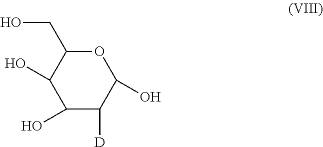 [SMILES: [2H]C1C(O)OC(CO)C(O)C1O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (VIII) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (VIII) or a pharmaceutically acceptable salt, solvate, hydrate, any isomer thereof, any metabolite thereof or physiologically functional derivative thereof.

In some examples, the compound is represented by Formula (IX):

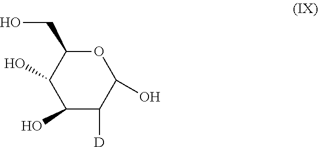 [SMILES: [2H]C1C(O)O[C@H](CO)[C@@H](O)[C@@H]1O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (IX) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (IX) or a pharmaceutically acceptable salt, solvate, hydrate, any isomer thereof, any metabolite thereof or physiologically functional derivative thereof.

In some examples, the compound is 2-deutero-2-deoxy-D-glucose.

In some examples, the compound is represented by Formula (X):

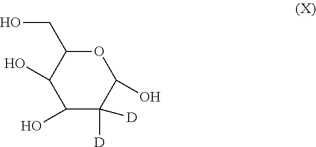 [SMILES: [2H]C1([2H])C(O)OC(CO)C(O)C1O]

or a pharmaceutically acceptable salt, solvate, hydrate, any isomer thereof, any tautomer thereof, any metabolite thereof or physiologically functional derivative thereof.

In some examples, the compound is 2-deutero-2-deoxy-D-glucose, also referred to as [2-D]2-deoxy-D-glucose or [2-²H]2-deoxy-D-glucose.

In some examples, the compound is or comprises [²H₂]2-deoxy-D-glucose and has the chemical name [2,2-D2]2-deoxy-D-glucose.

Hence, in accordance with some aspects, the present disclosure provides a compound for use in a method of acquiring at least one magnetic resonance (MR) spectrum and/or at least one MR image, wherein the compound is a represented by Formula (X) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (X) or a pharmaceutically acceptable salt, solvate, hydrate, any isomer thereof, any metabolite thereof or physiologically functional derivative thereof.

In some examples, the compound is represented by Formula (XI):

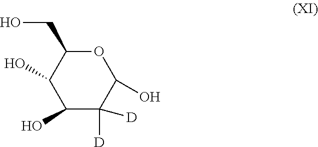 [SMILES: [2H]C1([2H])C(O)O[C@H](CO)[C@@H](O)[C@@H]1O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (XI) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (XI) or a pharmaceutically acceptable salt, solvate, hydrate, any isomer thereof, any metabolite thereof or physiologically functional derivative thereof.

In some embodiments, the compound represented by Formula (X) and (XI) are anomeric forms of [2,2-D2]2-deoxy-D-glucose.

In some embodiments, the compounds represented by Formulae (X) and (XI) are anomeric forms of [2,2-D2]2-deoxy-D-glucose.

In some examples, the compound is represented by Formula (XX):

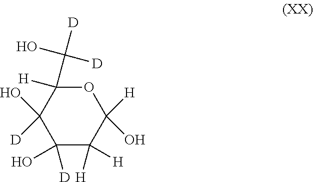 [SMILES: [2H]C([2H])(O)C1OC(O)CC([2H])(O)C1([2H])O]

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image wherein the compound is a represented by Formula (XX) or a pharmaceutically acceptable salt, solvate, hydrate, any isomer thereof, any metabolite thereof or physiologically functional derivative thereof.

In accordance with some other aspects, the present disclosure provides a compound for use in a method of diagnosing a subject, wherein said compound is represented by Formula (XX) or a pharmaceutically acceptable salt, solvate, hydrate, any isomer thereof, any metabolite thereof or physiologically functional derivative thereof.

In some examples, the compound is represented by Formula XXI.

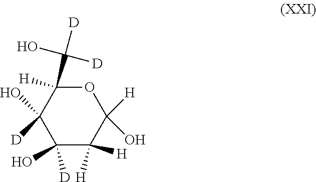

or a pharmaceutically acceptable salt, solvate, hydrate, any isomer thereof, any tautomer thereof, any metabolite thereof or physiologically functional derivative thereof.

Hence, in accordance with some aspects, the present disclosure provides a compound for use in a method of acquiring at least one magnetic resonance (MR) spectrum and/or at least one MR image, wherein the compound is a represented by Formula (XXI) or a pharmaceutically acceptable salt, solvate, hydrate, any isomer thereof, any metabolite thereof or physiologically functional derivative thereof.

In some embodiments, the compound represented by Formula (XX) and (XXI) are anomeric forms of [3,4,6,6-D4]2-deoxy-D-glucose.

In some embodiments, the compound represented by Formula (XX) and (XXI) refers to [3,4,6,6-D4]2-deoxy-D-glucose and includes α-anomeric forms, β-anomeric forms, mixtures thereof, solvates, hydrates, salts, and isotopically equivalent forms thereof unless the context indicates otherwise.

In accordance with some examples, the compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), (XX), (XXI) or a pharmaceutically acceptable salt, solvate, hydrate, any isomer thereof, any metabolite thereof or physiologically functional derivative thereof, or (f) a combination thereof.

The labeled compound described herein may comprise, an additional isotopically labeled atom, further to the labeled hydrogen atom. In some embodiments, the compound comprises at least one isotopically labeled nitrogen atom, ¹⁵N and/or at least one isotopically labeled carbon atom, ¹³C. In some embodiments, the compound consists of isotopically labeled hydrogen atoms. In some embodiments, the compound does not comprise other isotopically labeled atoms.

The isotopically substituted compound or compound of the present disclosure may be used in the form of a composition or a kit. Thus, the present disclosure further provides a composition comprising at least one compound detailed herein. It is noted that the composition may comprise at least one compound of the invention in a mixture with pharmaceutically acceptable auxiliaries, and optionally other therapeutic agents. The auxiliaries must be “acceptable” in the sense of being compatible with the other ingredients of the composition and not deleterious to the recipients thereof.

Compositions and compounds of the invention may be administered by any known method in the art. These include, but are not limited to, injection (e.g., using a subcutaneous, intramuscular, intravenous, intraarterial, or intradermal injection), dermal, intranasal administration, and oral administration. The amount of a compound according to the invention that may be used in a formulation of the invention, or generally administered to a subject, may be determined by the practitioner to provide an effective diagnosis, e.g., imaging.

Compositions administrable to a subject include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well known in the art of pharmacy. Such methods include the step of bringing in association a compound of the invention with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The present disclosure further provides a composition, as hereinbefore described, in combination with packaging material, including instructions for the use of the composition for diagnosis as detailed herein. For parenteral administration, suitable compositions include aqueous and non-aqueous sterile injection. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulizers or insufflators.

The present disclosure further provides a kit comprising at least one labeled compound of the invention and means for administering the at least one labeled compound and optionally instructions for use the kit in methods described herein.

As described above, 2DG compounds including the isotopically substituted 2DG compound described herein do not have a hydroxyl at C-2 as this hydroxyl is replaced by a hydrogen atom (in 2DG) or with a deuterium atom in one or more of the compounds described herein. The hydroxyl replacement (either to a hydrogen atom or a deuterium atom) does not interfere with the first step of glycolysis and hence 2DG compound or the labeled compound undergo phosphorylation mediated by hexokinase at the C-6 hydroxyl to 2DG 6-phosphate and/or deuterated 2DG 6-phosphate (at times phosphorylated product). However, further metabolism of 2DG 6-phosphate and/or deuterated 2DG 6-phosphate to D-fructose 6-phosphate is not possible.

The phosphorylated product of any of the compounds described herein is denoted as a metabolite of the 2DG isotopically substituted/labeled compound.

In some examples, the metabolite is at least one of [²H]2-deoxy-D-glucose-6-phosphate, [²H₂]2-deoxy-D-glucose-6-phosphate, [²H₃]2-deoxy-D-glucose-6-phosphate, [²H₄]2-deoxy-D-glucose-6-phosphate, [²H₅]2-deoxy-D-glucose-6-phosphate, [²H₆]2-deoxy-D-glucose-6-phosphate, [²H₇]2-deoxy-D-glucose-6-phosphate, [²H₈]2-deoxy-D-glucose-6-phosphate or a combination thereof.

In some examples, the metabolite is [²H]2-deoxy-D-glucose-6-phosphate. In some examples, the metabolite is [²H₂]2-deoxy-D-glucose-6-phosphate. In some examples, the metabolite is [²H₃]2-deoxy-D-glucose-6-phosphate. In some examples, the metabolite is [²H₄]2-deoxy-D-glucose-6-phosphate. In some examples, the metabolite is [²H₅]2-deoxy-D-glucose-6-phosphate. In some examples, the metabolite is [²H₆]2-deoxy-D-glucose-6-phosphate. In some examples, the metabolite is [²H₇]2-deoxy-D-glucose-6-phosphate. In some examples, the metabolite is [²H₈]2-deoxy-D-glucose-6-phosphate.

In some examples, the metabolite is a phosphorylation product of a compound represented by Formula (I). In some examples, the metabolite is a phosphorylation product of a compound represented by Formula (II). In some examples, the metabolite is a phosphorylation product of a compound represented by Formula (III). In some examples, the metabolite is a phosphorylation product of a compound represented by Formula (IV). In some examples, the metabolite is a phosphorylation product of a compound represented by Formula (V). In some examples, the metabolite is a phosphorylation product of a compound represented by Formula (VI). In some examples, the metabolite is a phosphorylation product of a compound represented by Formula (VII). In some examples, the metabolite is a phosphorylation product of a compound represented by Formula (VIII). In some examples, the metabolite is a phosphorylation product of a compound represented by Formula (IX). In some examples, the metabolite is a phosphorylation product of a compound represented by Formula (X). In some examples, the metabolite is a phosphorylation product of a compound represented by Formula (XI). In some examples, the metabolite is a phosphorylation product of a compound represented by Formula (XX). In some examples, the metabolite is a phosphorylation product of a compound represented by Formula (XXI).

In accordance with some examples, the metabolite is represented by Formula (XII):

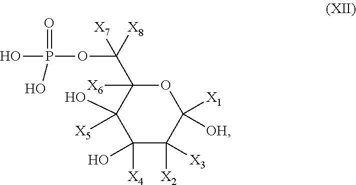

or a pharmaceutically acceptable salt, solvate, hydrate, any isomer thereof, any tautomer thereof, any metabolite thereof or physiologically functional derivative thereof, wherein at least one of X₁, X₂, X₃, X₄, X₅, X₆, X₇, X₈ is ²H (D).

In accordance with some examples, the metabolite is represented by Formula (XIII):

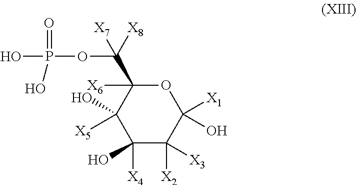

or a pharmaceutically acceptable salt, solvate, hydrate, any isomer thereof, any tautomer thereof, any metabolite thereof or physiologically functional derivative thereof, wherein at least one of X₁, X₂, X₃, X₄, X₅, X₆, X₇, X₈ is ²H (D).

In accordance with some examples, the metabolite is represented by Formula (XIV):

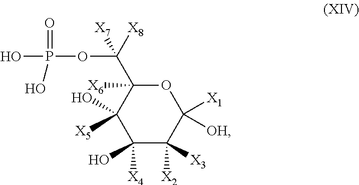

or a pharmaceutically acceptable salt, solvate, hydrate, any isomer thereof, any tautomer thereof, any metabolite thereof or physiologically functional derivative thereof, wherein at least one of X₁, X₂, X₃, X₄, X₅, X₆, X₇, X₈ is ²H (D).

In accordance with some examples, the metabolite is represented by Formula (XV):

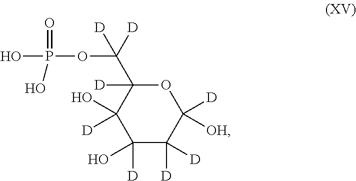 [SMILES: [2H]C([2H])(OP(=O)(O)O)C1([2H])OC([2H])(O)C([2H])([2H])C([2H])(O)C1([2H])O]

or a pharmaceutically acceptable salt, solvate, hydrate, any isomer thereof, any tautomer thereof, any metabolite thereof or physiologically functional derivative thereof.

In accordance with some examples, the metabolite is represented by Formula (XVI):

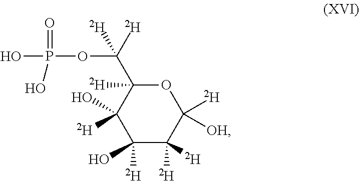

or a pharmaceutically acceptable salt, solvate, hydrate, any isomer thereof, any tautomer thereof, any metabolite thereof or physiologically functional derivative thereof.

In some examples, the metabolite is or comprises [²H₂]2-deoxy-D-glucose-6-phosphate.

In accordance with some examples, the metabolite is represented by Formula (XVII):

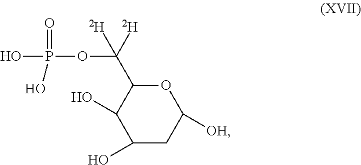 [SMILES: [2H]C([2H])(OP(=O)(O)O)C1OC(O)CC(O)C1O]

or a pharmaceutically acceptable salt, solvate, hydrate, any isomer thereof, any tautomer thereof, any metabolite thereof or physiologically functional derivative thereof.

In accordance with some examples, the metabolite is represented by Formula (XVIII):

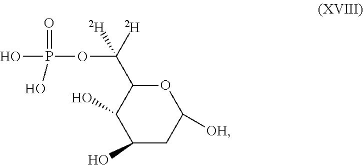

or a pharmaceutically acceptable salt, solvate, hydrate, any isomer thereof, any tautomer thereof, any metabolite thereof or physiologically functional derivative thereof.

In some examples, the metabolite is or comprises [²H₄]2-deoxy-D-glucose-6-phosphate.

In accordance with some examples, the metabolite is represented by Formula (XXII):

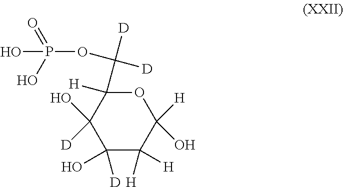 [SMILES: [2H]C([2H])(OP(=O)(O)O)C1OC(O)CC([2H])(O)C1([2H])O]

or a pharmaceutically acceptable salt, solvate, hydrate, any isomer thereof, any tautomer thereof, any metabolite thereof or physiologically functional derivative thereof.

In accordance with some examples, the metabolite is represented by Formula (XXIII):

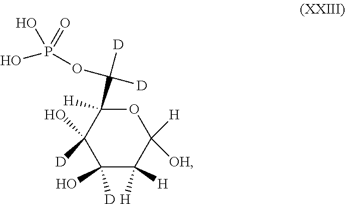

or a pharmaceutically acceptable salt, solvate, hydrate, any isomer thereof, any tautomer thereof, any metabolite thereof or physiologically functional derivative thereof.

In some embodiments that may be considered as aspects of the invention, the compound of the present disclosure as well as a metabolite thereof is for use in a method of diagnosis a subject. In some embodiments that may be considered as aspects of the invention, the 2DG labeled compound and/or a metabolite thereof is for use in a method of diagnosis a condition or a disease in a subject.

In some embodiments that may be considered as aspects of the invention, the 2DG labeled compound and/or a metabolite thereof is for use in a method of monitoring a disease state in the subject.

In some embodiments that may be considered as aspects of the invention, the 2DG labeled compound and/or a metabolite thereof is for use in a method of determining a site of a disease in the subject.

In some embodiments that may be considered as aspects of the invention, the 2DG labeled compound and/or a metabolite thereof is for use in a method of (i) diagnosis a condition or a disease in a subject, (ii) monitoring a disease state in the subject or (iii) determining a site of a disease in the subject, wherein the 2DG labeled compound is at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof and wherein the metabolite is at least one of [²H]2-deoxy-D-glucose-6-phosphate, [²H₂]2-deoxy-D-glucose-6-phosphate, [²H₃]2-deoxy-D-glucose-6-phosphate, [²H₄]2-deoxy-D-glucose-6-phosphate, [²H₅]2-deoxy-D-glucose-6-phosphate, [²H]2-deoxy-D-glucose-6-phosphate, [²H₇]2-deoxy-D-glucose-6-phosphate, [²H₈]2-deoxy-D-glucose-6-phosphate or a combination thereof.

In some embodiments that can be considered as aspects of the present disclosure, the 2DG labeled compound and/or a metabolite thereof is for use in a method of (i) diagnosis a condition or a disease in a subject, (ii) monitoring a disease state in the subject or (iii) determining a site of a disease in the subject, wherein the 2DG labeled compound is [²H₈]2-deoxy-D-glucose and the metabolite is or comprises [²H₈]2-deoxy-D-glucose-6-phosphate.

In some embodiments that can be considered as aspects of the present disclosure, the 2DG labeled compound and/or a metabolite thereof is for use in a method of (i) diagnosis a condition or a disease in a subject, (ii) monitoring a disease state in the subject or (iii) determining a site of a disease in the subject, wherein the 2DG labeled compound is [²H₄]2-deoxy-D-glucose and the metabolite is or comprises [²H₄]2-deoxy-D-glucose-6-phosphate.

In some embodiments that can be considered as aspects of the present disclosure, the 2DG labeled compound and/or a metabolite thereof is for use in a method of (i) diagnosis a condition or a disease in a subject, (ii) monitoring a disease state in the subject or (iii) determining a site of a disease in the subject, wherein the 2DG labeled compound is [3,4,6,6-D4]2-deoxy-D-glucose and the metabolite is or comprises [3,4,6,6-D4]2-deoxy-D-glucose-6-phosphate.

As described herein, the compounds of the disclosure are applicable to use in MR methods such as MRS and/or MRI.

In accordance with some aspects, it is provided a method of acquiring at least one MR spectrum and/or MR image from a subject, the method comprises monitoring a signal from a subject using an MR method, the subject having been administered at least one compound, a composition or a kit comprising the at least one compound to thereby acquire at least one MR image, wherein the compound is an isotopically substituted 2DG compound.

MRS and MRI methods make use of magnetic fields, radio waves, and in case of MRI also field gradients to generate at least one spectrum or at least one image of the organs in the body. The methods of the invention are applicable by using a magnetic resonance scanner (an MRI scanner). Magnetic resonance signals obtained by the methods described herein may be converted by conventional manipulations into 2-, 3- or 4-dimensional data (spatial and temporal) including metabolic, kinetic, diffusion, relaxation, and physiological data. Magnetic resonance spectroscopy may be conducted by any suitable probe, for example using a ²H Radio Frequency coil.

Detecting such signal using MR can be done by using for example specialized probes for acquiring the signal. For example, diagnosis of breast cancer may be done by subjecting a subject to magnetic field and using specific MR sequences and equipment (e.g. probes) to acquire signal from the breast region of a subject.

In another example, diagnosis of an ischemic tissue in the brain or body may be done by subjecting a subject to magnetic field and using specific MR sequences and equipment (e.g. probes, i.e. radio-frequency circuits, i.e. radio-frequency coils) to acquire signal from the brain or the body region of a subject.

In accordance with some aspects, it is provided a method of imaging a subject, the method comprises monitoring a signal from a subject using an MR imaging method, the subject having been administered at least one compound, a composition or the at least one compound comprised in a kit, to thereby acquire at least one MR image, wherein the compound is deuterium substituted 2DG compound.

In accordance with some aspects, it is provided a method of imaging a subject, the method comprises monitoring a signal from a subject using an MR imaging method, the subject having been administered at least one compound, a composition or the at least one compound comprised in a kit, to thereby acquire at least one MR image of the at least one compound or a metabolite thereof, wherein the compound is deuterium labeled 2DG.

In some examples, in the methods described herein, the subject has been administered with at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof. In some examples, the subject has been administered with [D₈]2-deoxy-D-glucose.

In some examples, in the methods described herein, the subject has been administered with [²H]2-deoxy-D-glucose.

In some examples, in the methods described herein, the subject has been administered with [²H₂]2-deoxy-D-glucose. In some examples, in the methods described herein, the subject has been administered with [²H₃]2-deoxy-D-glucose. In some examples, in the methods described herein, the subject has been administered with [²H₄]2-deoxy-D-glucose. In some examples, the subject has been administered with [3,4,6,6-D4]2-deoxy-D-glucose. In some examples, in the methods described herein, the subject has been administered with [²H₅]2-deoxy-D-glucose. In some examples, in the methods described herein, the subject has been administered with [²H₆]2-deoxy-D-glucose. In some examples, in the methods described herein, the subject has been administered with [²H₇]2-deoxy-D-glucose. In some examples, in the methods described herein, the subject has been administered with [²H₈]2-deoxy-D-glucose. In some examples, in the methods described herein, the subject has been administered with at least one compound represented by Formula (I) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), a composition or the at least one compound comprised in a kit.

In some examples, in the methods described herein the subject has been administered with at least one compound represented by Formula (II) or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), a composition or the at least one compound comprised in a kit.

In some examples, in the methods described herein the subject has been administered at least one compound represented by Formula (I) or Formula (I′) and/or Formula (II), and/or Formula (II′), and/or Formula (III), and/or Formula (III′), in which one, two, three, four, five, six, seven or eight of X₁, X₂, X₃, X₄, X₅, X₆, X₇, and X₈ is ²H.

In some examples, in the methods described herein the subject has been administered at least one compound represented by Formula (IV)-Formula (XI), Formula (XX), Formula (XXI) or a pharmaceutically acceptable salt, solvate, hydrate, any isomer thereof, any metabolite thereof or physiologically functional derivative thereof.

In some examples, in the methods described herein the subject has been administered at least one compound represented by Formula (IV), or Formula (V) or a pharmaceutically acceptable salt, solvate, hydrate, any isomer thereof, any metabolite thereof or physiologically functional derivative thereof.

In some examples, in the methods described herein the subject has been administered at least one compound represented by Formula (VI), or Formula (VII) or a pharmaceutically acceptable salt, solvate, hydrate, any isomer thereof, any metabolite thereof or physiologically functional derivative thereof.

In some examples, the subject has been administered at least one compound represented by Formula (VIII), or Formula (IX) or a pharmaceutically acceptable salt, solvate, hydrate, any isomer thereof, any metabolite thereof or physiologically functional derivative thereof.

In some examples, the subject has been administered at least one compound represented by Formula (X), or Formula (XI) or a pharmaceutically acceptable salt, solvate, hydrate, any isomer thereof, any metabolite thereof or physiologically functional derivative thereof.

In some examples, the subject has been administered at least one compound represented by Formula (XX), or Formula (XXI) or a pharmaceutically acceptable salt, solvate, hydrate, any isomer thereof, any metabolite thereof or physiologically functional derivative thereof.

In another aspect, the invention provides a method for imaging at least one body region of a subject, the method comprising (i) administering to the subject an effective amount of a deuterium labeled 2DG, a composition or a kit comprising the at least one compound and (ii) imaging at least one body region. In some embodiments, the method comprises imaging full body. It should be noted that full body imaging (also termed as whole-body imaging) refers to display of the entire body in a single imaging examination.

In some embodiments, following administration of a deuterium-labeled 2DG compound described herein, the method comprises acquiring at least one deuterium (²H) MR image of the subject, or of one or more body regions of the subject. In some embodiments, the acquired ²H image is configured to depict the distribution, uptake, localization, accumulation, retention, clearance, and/or metabolism of the administered deuterium-labeled 2DG compound and/or one or more metabolites thereof in the subject's tissues.

In some embodiments, the ²H image is acquired from the whole body of the subject.

In some embodiments, the ²H image is acquired from one or more body regions, organs, lesions, tumors, disease sites, or regions of interest. In some embodiments, the acquired image is used to assess uptake, biodistribution, accumulation, or metabolism of the administered deuterium-labeled 2DG compound and/or one or more metabolites thereof in normal tissue and/or diseased tissue.

In some embodiments, the imaging methods comprises monitoring a signal from a subject using an MR imaging method, wherein the compound is at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof.

In some embodiments, the imaging method comprises monitoring a signal from a subject using an MR imaging method, wherein the subject has been administered with a compound being [D₈]2-deoxy-D-glucose.

In some embodiments, the imaging method comprises monitoring a signal from a subject using an MR imaging method, wherein the subject has been administered with a compound being [D₄]2-deoxy-D-glucose. In some embodiments, the imaging method comprises monitoring a signal from a subject using an MR imaging method, wherein the subject has been administered with a compound being [3,4,6,6-D4]2-deoxy-D-glucose, In some embodiments, the imaging methods comprise monitoring a signal from a subject using an MR imaging method, wherein subject has been administered with at least one compound represented by Formula (I) or Formula (I′) and/or Formula (II), and/or Formula (II′), and/or Formula (III), and/or Formula (III′), in which one, two, three, four, five, six, seven or eight of X₁, X₂, X₃, X₄, X₅, X₆, X₇, and X₈ is ²H.

In some embodiments, the imaging methods comprise monitoring a signal from a subject using an MR imaging method, wherein subject has been administered with at least one compound represented by Formula (IV)-Formula (XI), (XX), (XXI) or a pharmaceutically acceptable salt, solvate, hydrate, any isomer thereof, any metabolite thereof or physiologically functional derivative thereof.

In some embodiments, the imaging methods comprise monitoring a signal from a subject using an MR imaging method, wherein subject has been administered with at least one compound represented by Formula (IV), or Formula (V) or a pharmaceutically acceptable salt, solvate, hydrate, any isomer thereof, any metabolite thereof or physiologically functional derivative thereof.

In some embodiments, the imaging methods comprise monitoring a signal from a subject using an MR imaging method, wherein subject has been administered with at least one compound represented by Formula (VI), or Formula (VII) or a pharmaceutically acceptable salt, solvate, hydrate, any isomer thereof, any metabolite thereof or physiologically functional derivative thereof.

In some embodiments, the imaging methods comprise monitoring a signal from a subject using an MR imaging method, wherein subject has been administered with at least one compound represented by Formula (VIII), or Formula (IX) or a pharmaceutically acceptable salt, solvate, hydrate, any isomer thereof, any metabolite thereof or physiologically functional derivative thereof.

In some embodiments, the imaging methods comprise monitoring a signal from a subject using an MR imaging method, wherein subject has been administered with at least one compound represented by Formula (X), or Formula (XI) or a pharmaceutically acceptable salt, solvate, hydrate, any isomer thereof, any metabolite thereof or physiologically functional derivative thereof.

In some embodiments, the imaging methods comprise monitoring a signal from a subject using an MR imaging method, wherein the subject has been administered with at least one compound represented by Formula (XX), or Formula (XXI) or a pharmaceutically acceptable salt, solvate, hydrate, any isomer thereof, any metabolite thereof or physiologically functional derivatives thereof.

As described herein, the methods may include a monitoring step.

As described herein, monitoring and detecting a signal from the compounds and/or a metabolite thereof for the invention may be performed by any non-invasive method, preferably, as detailed herein, by MRS, MRI and magnetic resonance spectroscopic imaging.

In some embodiments, the monitoring methods described herein comprise one or more magnetic resonance acquisition strategies configured to facilitate detection, visualization, localization, quantification, mapping, characterization, or assessment of a deuterium-labeled compound described herein and/or one or more metabolites thereof. The acquisition strategies may exploit one or more magnetic resonance properties of the administered compound and/or metabolites thereof, including resonance frequency, chemical shift, relaxation behavior, saturation behavior, recovery behavior, or combinations thereof.

In some embodiments, the magnetic resonance acquisition is configured to exploit one or more differences between a deuterium-labeled compound described herein and naturally occurring deuterium-containing species, including differences in resonance frequency, chemical shift, longitudinal relaxation time (T1), transverse relaxation time (T2), effective transverse relaxation time (T2*), saturation behavior, recovery behavior, exchange behavior, diffusion properties, or combinations thereof.

In some embodiments, the magnetic resonance acquisition is configured such that signal arising from the administered deuterium-labeled 2DG compound and/or one or more metabolites thereof is detected preferentially relative to signal arising from naturally occurring deuterium-containing species present in the subject, including deuterium in water (HDO). In some embodiments, the acquired ²H image comprises a reduced, attenuated, suppressed, or otherwise decreased contribution from naturally occurring HDO relative to the contribution of the administered deuterium-labeled 2DG compound and/or one or more metabolites thereof.

In some embodiments, such preferential detection is achieved by exploiting one or more MR properties that differ between the administered deuterium-labeled 2DG compound and/or one or more metabolites thereof, on the one hand, and naturally occurring deuterium-containing species including HDO, on the other hand. In some embodiments, the acquisition exploits a difference in longitudinal relaxation time (T1).

In some embodiments, one or more deuterons of the administered deuterium-labeled 2DG compound and/or one or more metabolites thereof exhibit a shorter T1 than deuterium in HDO under the relevant acquisition conditions, thereby permitting preferential detection of the administered compound and/or metabolite relative to HDO.

In some embodiments, the acquisition comprises selection of one or more imaging parameters including repetition time (TR), echo time (TE), inversion time (TI), flip angle, excitation pulse characteristics, spectral bandwidth, spectral resolution, acquisition duration, number of dummy scans, number of averages, spatial resolution, temporal resolution, or combinations thereof, to improve detection of the administered deuterium-labeled compound and/or one or more metabolites thereof.

In some embodiments, the acquisition is performed using one or more pulse sequence parameters selected to take advantage of such relaxation differences, including repetition time (TR), inversion time (TI), flip angle, saturation pulses, inversion pulses, preparation pulses, recovery delays, dummy scans, steady-state preparation, or combinations thereof. In some embodiments, repeated excitation using a relatively short TR results in greater saturation of HDO-derived signal than of signal arising from the administered deuterium-labeled 2DG compound and/or one or more metabolites thereof.

In some embodiments, one or more dummy scans are used before image acquisition to establish a magnetization state in which signal from naturally occurring HDO is attenuated relative to signal from the administered compound and/or one or more metabolites thereof. In some embodiments, an inversion recovery acquisition is used, optionally with selection of an inversion time (TI) that reduces, attenuates, suppresses, or substantially nulls signal from naturally occurring HDO while preserving detectable signal from the administered deuterium-labeled 2DG compound and/or one or more metabolites thereof.

In some embodiments, magnetic resonance imaging of a deuterium-labeled compound and/or a metabolite thereof is performed using a chemical shift imaging (CSI) sequence, a magnetic resonance spectroscopic imaging (MRSI) sequence, a spectroscopic sequence, a gradient echo sequence, a spoiled gradient echo sequence, a fast low-angle shot (FLASH) sequence, an echo-planar imaging (EPI) sequence, a balanced steady-state free precession (bSSFP) sequence, a steady-state free precession (SSFP) sequence, a spin echo sequence, a fast spin echo sequence, a turbo spin echo sequence, a rapid acquisition with relaxation enhancement (RARE) sequence, a projection imaging sequence, a three-dimensional imaging sequence, a single-shot sequence, a multi-shot sequence, an inversion recovery sequence, a saturation recovery sequence, a magnetization-prepared sequence, or a combination thereof.

In some embodiments, the acquisition comprises one or more saturation pulses, inversion pulses, preparation pulses, recovery periods, magnetization transfer pulses, spectral-selective pulses, frequency-selective pulses, water-suppression pulses, deuterium-suppression pulses, or combinations thereof.

In some embodiments, imaging is performed using a T1-weighted acquisition, a T2-weighted acquisition, a T2*-weighted acquisition, an inversion recovery sequence, a saturation recovery sequence, a steady-state sequence, a magnetization-prepared sequence, or a combination thereof.

In some embodiments, the magnetic resonance acquisition is configured to selectively enhance, attenuate, suppress, or distinguish signals arising from different deuterium-containing species. Such selectivity may be achieved by exploiting differences in longitudinal relaxation time (T1), transverse relaxation time (T2), effective transverse relaxation time (T2*), saturation characteristics, recovery characteristics, or combinations thereof.

As used herein, an “inversion recovery sequence” refers to a magnetic resonance acquisition sequence in which an inversion pulse is applied prior to signal acquisition to invert longitudinal magnetization, followed by a recovery period before excitation and signal detection. The duration of the recovery period, optionally referred to as the inversion time (TI), may be selected to enhance, attenuate, suppress, null, distinguish, or otherwise modulate signals arising from one or more compounds, tissues, metabolites, or nuclei based on differences in longitudinal relaxation time (T1).

In some embodiments, an inversion recovery sequence is configured to attenuate, suppress, null, reduce, or otherwise distinguish signals originating from naturally occurring deuterium-containing species, including HDO, relative to signals originating from an administered deuterium-labeled compound and/or one or more metabolites thereof. In some embodiments, the inversion time (TI) is selected based on differences in longitudinal relaxation time (T1) between the naturally occurring deuterium-containing species and the administered deuterium-labeled compound and/or one or more metabolites thereof such that the magnetization of the naturally occurring deuterium-containing species is substantially reduced or nulled at the time of signal acquisition while the administered deuterium-labeled compound and/or one or more metabolites thereof remain detectable.

In some embodiments, a method comprises administering to a subject a deuterium-labeled 2DG compound, wherein one or more deuterium atoms are directly bound to carbon atoms of the 2DG molecule, and acquiring at least one ²H MR image from the subject using an acquisition sequence configured to preferentially detect signal from said deuterium-labeled 2DG compound and/or a metabolite thereof relative to naturally occurring deuterium in body water (HDO), wherein said preferential detection exploits a shorter longitudinal relaxation time (T1) of deuterium nuclei bound to carbon atoms of said compound relative to the T1 of deuterium in HDO. In some embodiments, the acquisition sequence comprises one or more of a short repetition time (TR), dummy scans, an inversion recovery component, or a saturation component selected to attenuate HDO-derived signal while preserving detectable signal from the administered compound and/or one or more metabolites thereof. In some embodiments, the T1 of deuterium nuclei directly bound to carbon atoms of the 2DG molecule is between about 5 milliseconds and about 200 milliseconds, which is significantly shorter than the T1 of deuterium in HDO, as demonstrated in Table 1, thereby enabling preferential detection of the administered compound and/or metabolite relative to background HDO signal.

While the methods described herein may employ a variety of magnetic resonance acquisition sequences, in some embodiments the acquisition comprises a spectroscopic imaging approach configured to spatially localize signals originating from a deuterium-labeled compound and/or one or more metabolites thereof.

In some embodiments, magnetic resonance imaging of a deuterium-labeled compound and/or a metabolite thereof is performed using a chemical shift imaging (CSI) sequence, also referred to as magnetic resonance spectroscopic imaging (MRSI). As used herein, “chemical shift imaging” refers to an MR acquisition technique that combines spatial encoding and spectral encoding to generate spatially resolved maps representing the distribution of one or more MR-detectable compounds based on their respective resonance frequencies.

In some embodiments, the MR acquisition comprises acquisition of deuterium (²H) signals following administration of a deuterium-labeled compound described herein. In some embodiments, the acquisition is performed using a repetition time (TR) of less than about 100 ms, less than about 75 ms, less than about 50 ms, less than about 25 ms, less than about 20 ms, less than about 16 ms, or any value or range therebetween. In some embodiments, the acquisition comprises a plurality of spectral acquisition points and a spectral bandwidth sufficient to separate signals originating from the administered deuterium-labeled compound from signals originating from naturally occurring deuterium-containing species, or combinations.

In some embodiments, one or more non-acquisition scans (“dummy scans”) are performed prior to acquisition of imaging data. As used herein, a “dummy scan” refers to an excitation cycle that is not included in image reconstruction and is performed to establish a desired magnetization state before data acquisition. In some embodiments, the dummy scans establish a steady-state magnetization condition, produce saturation of one or more background signals, suppress naturally occurring deuterium-containing species, attenuate HDO-derived signals, or combinations thereof.

Without wishing to be bound by theory, naturally occurring deuterium present in body water (HDO) may exhibit a longer longitudinal relaxation time (T1) than a deuterium-labeled compound described herein and/or one or more metabolites thereof (for example as shown in Table 1). Consequently, repeated excitation pulses, particularly when applied using a short repetition time (TR), may result in incomplete longitudinal magnetization recovery for HDO and progressive saturation of the HDO signal. In contrast, the administered deuterium-labeled compound and/or metabolites thereof may recover magnetization more rapidly and remain detectable under the same acquisition conditions. One or more dummy scans may further establish such differential saturation before image acquisition. Accordingly, signals arising from naturally occurring HDO may be selectively attenuated relative to signals arising from the administered deuterium-labeled compound and/or one or more metabolites thereof, thereby improving signal-to-noise ratio, contrast, detection sensitivity, specificity, quantification accuracy, spatial localization, temporal resolution, acquisition speed, image quality, or combinations thereof.

In some embodiments, the acquisition approaches described herein improve one or more of signal-to-noise ratio, image contrast, specificity for the administered compound and/or metabolite, detection sensitivity, quantification accuracy, spatial resolution, temporal resolution, acquisition speed, and clinical practicality. In some embodiments, preferential attenuation of naturally occurring HDO-derived signal facilitates acquisition of deuterium images that more accurately reflect tissue distribution or uptake of the administered deuterium-labeled 2DG compound and/or one or more metabolites thereof.

In some embodiments, the methods described herein permit visualization, detection, monitoring, localization, quantification, mapping, tracking, or assessment of uptake, accumulation, retention, clearance, metabolism, biodistribution, pharmacokinetics, disease progression, treatment response, or combinations thereof by imaging a deuterium-labeled compound and/or one or more metabolites thereof in a tissue, organ, lesion, tumor, disease site, or region of interest. In some embodiments, imaging is performed at one or more time points following administration of the deuterium-labeled compound, including seconds, minutes, hours, days, or combinations thereof after administration.

In some embodiments, at least one ²H image is acquired at one or more time points after administration of the deuterium-labeled 2DG compound, including seconds, minutes, or hours after administration. In some embodiments, a plurality of ²H images is acquired over time to monitor uptake, retention, metabolism, clearance, disease progression, or treatment response.

It should be noted that monitoring a signal encompasses collecting data, possibly in the form of an MR image or an MR spectrum, from a subject, including any region of the subject's body. The term “monitoring” as used herein as part of the MR method is meant to encompass the quantitative and/or qualitative detection and observation of a signal originating from the compound of the invention being administered to the subject or a metabolite thereof as described herein. Monitoring of the signal originating from the compound of the invention encompasses monitoring/detecting the distribution of the compound in at least one body region and/or full body as further described herein, wherein the method comprises acquiring at least one ²H image from the subject's body or one or more body regions after the administration step. As noted, and in some examples, the image represented solely or preferentially the 2DG distribution or uptake in the subject's body tissues and less preferentially the natural abundance of deuterium in water (HDO), whereas obtaining such an image relies on the shorter T1 of the 2DG deuterons compared to HDO or linear molecules.

It should be noted that as described herein, monitoring may encompass quantitative and/or qualitative detection and observation of a signal obtained prior to administration of a compound of the invention.

In some examples, the MR method comprises monitoring uptake of the deuterated labeled compound of the present disclosure. In some examples, the MR method comprises monitoring accumulation of the deuterated labeled metabolite of the present disclosure.

In some examples, the method comprises monitoring uptake of the deuterated labeled compound of the present disclosure by acquiring at least one ²H MR image from a subject. In some examples, the MR method comprises monitoring accumulation of the deuterated labeled metabolite of the present disclosure by acquiring at least one ²H MR image from a subject.

In some examples, the MR method comprises monitoring accumulation of the deuterated labeled metabolite by acquiring at least one ²H MR image from a subject, wherein the labeled metabolite is at least one of [²H]2-deoxy-D-glucose-6-phosphate, [²H₂]2-deoxy-D-glucose-6-phosphate, [²H₃]2-deoxy-D-glucose-6-phosphate, [²H₄]2-deoxy-D-glucose-6-phosphate, [²H₅]2-deoxy-D-glucose-6-phosphate, [²H₆]2-deoxy-D-glucose-6-phosphate, [²H₇]2-deoxy-D-glucose-6-phosphate, [²H₈]2-deoxy-D-glucose-6-phosphate or a combination thereof.

The chemical shift of the 2DG and the metabolite may be the same.

In some examples, the MR method comprises monitoring accumulation of the deuterated labeled metabolite by acquiring at least one ²H MR image from a subject, wherein the metabolite is or comprises [²H]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₂]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₃]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₄]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₅]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₆]2-deoxy-D-glucose-6-phosphate.

In some examples, the MR method comprises monitoring accumulation of the deuterated labeled metabolite by acquiring at least one ²H MR image from a subject, wherein the metabolite is or comprises [²H₇]2-deoxy-D-glucose-6-phosphate.

In some examples, the MR method comprises monitoring accumulation of the deuterated labeled metabolite by acquiring at least one ²H MR image from a subject, wherein the labeled metabolite is or comprises [²H₈]2-deoxy-D-glucose-6-phosphate.

In some examples, the MR method comprises monitoring accumulation of the deuterated labeled metabolite by acquiring at least one ²H MR image from a subject, wherein the metabolite is a phosphorylation product of a compound represented by one or more of Formula (I), Formula (II), Formula (III), Formula (IV), Formula (V), Formula (VI), Formula (VII), Formula (VIII), Formula (IX), Formula (X), Formula (XI), Formula (XX), Formula (XXI), or any combination thereof.

In some examples, the MR method comprises monitoring accumulation of the deuterated labeled metabolite by acquiring at least one ²H MR image from a subject, wherein the labeled metabolite is represented by Formula (XII)-(XVIII), (XXII) and (XXIII).

The magnetic resonance acquisition strategies described herein may, in some embodiments, benefit from the relaxation characteristics of the deuterium-labeled compounds and metabolites described herein. Accordingly, the inventors investigated relaxation-related properties that may influence image acquisition parameters, signal recovery, acquisition speed, signal-to-noise ratio, image contrast, spatial resolution, temporal resolution, and overall detectability.

As appreciated, MR imaging rely on averaging, i.e., accumulating the same data multiple times for improving the image signal-to-noise ratio (SNR), that is often required to enable high spatiotemporal resolution.

The relaxation times of the sampled atom, e.g. deuterons or protons, respectively, specifically their T₂*, T₂, and T₁, limit and dictate the MRI acquisition parameters and the number of averages that can be acquired per unit time.

The term relaxation describes how signals change with time. In general signals deteriorate with time, becoming weaker and broader. The deterioration reflects the fact that the MR signal, which results from nuclear magnetization, arises from the over-population of an excited state. Relaxation is the conversion of this non-equilibrium population to a normal population. In other words, relaxation describes how quickly spins “forget” the direction in which they are oriented. The deterioration of an MR signal is analyzed in terms of two separate processes, each with their own time constants. One process, associated with T₁, is responsible for the loss of signal intensity. Thus, the effective lifetime of the compound is dictated by the compound's T₁ relaxation.

The free induction decay (FID) of the deuterium signal of the species with the longest T₂*, T₂ or T₁ will, in principle, limit the repetition time and the number of averages that can be recorded per unit time in a manner that will be dependent on the pulse sequence used for recording the image. The relaxation times will always, from first principles, follow the order T₁>T₂>T₂*, i.e., T₁ is always the longest relaxation time constant.

Without wishing to be bound by theory, the inventors propose that the relaxation characteristics of deuterium nuclei associated with the compounds described herein may contribute to the effectiveness of the acquisition strategies described above, including short repetition time acquisitions, steady-state acquisitions, saturation-based acquisitions, inversion recovery acquisitions, dummy-scan based acquisitions, and combinations thereof.

As shown in Table 1 in the examples that form a part of this application, T₁ of deuterium nuclei attached to a ring carbon (including sugars) is significantly shorter (about 6- to 10-fold) than that of deuterium nuclei in water (HDO). Hence, it was suggested that it is possible to use compounds having ²H directly bound to carbon atoms and hence to decrease (reduce) the compound's T₁. In this way, it has been suggested by the inventors to dramatically reduce the MR repetition and acquisition time, while allowing for preferential observation of the 2DG compounds rather than the HDO signals, allowing acquisition of multiple MR scans, hence improving SNR and improving clinical conclusions.

Based on these findings, it was suggested that the application of 2DG compounds labelled with one or more deuterium atoms as described herein, allow on one hand very fast deuterium imaging (for example in the range of seconds or minutes, e.g. about 30 seconds, about 1 minute, about 2 minutes) with high SNR and on the other hand that such images will show preferentially the 2DG compounds' distribution and less preferentially the distribution of HDO. It should be noted that application of [6,6-D₂]D-glucose is often challenging due to the need of a long acquisition time, usually more than 15 minutes.

Without being bound by theory, it was suggested that the T₁ relaxation time of the 2DG deuterated compound, for example [²H₈]2-deoxy-D-glucose, allow reduction of the acquisition time of the images and increase the images spatiotemporal resolution. These advantages make the compounds and acquisition approaches described herein to be clinically practical.

In some embodiments, the compound is characterized by a T₁ relaxation of a ²H nucleus of between about 5 milliseconds to about 400 milliseconds, at times between about 5 milliseconds and about 300 milliseconds, at times between about 5 milliseconds and about 200 milliseconds, at times between about 5 milliseconds and about 100 milliseconds, at times between about 5 milliseconds and about 60 millisecond.

In some examples, the methods comprise acquiring at least one MR spectrum from a subject. In some examples, the method comprises acquiring at least one ²H MR spectrum from a subject.

The methods described herein may be of value as they reduce the acquisition time of a deuterium image and increase the signal to noise ratio of a deuterium image. It was suggested that the methods described herein may reduce/attenuate/decrease the signal acquired from naturally abundant signals of deuterium in water (HDO).

In some examples, the method comprises acquiring at least one MR image from a subject. In some examples, the method comprises acquiring at least one ²H MR image from a subject. In some examples, the method comprises acquiring at least one ²H MR image from a subject with an acquisition time of at most about 5 min, at most about 4 min., at times at most about 2 min. In some examples, the method comprises acquiring at least one ²H MR image from a subject with an in-plane spatial resolution of between about 2 mm and about 20 mm, at times between about 2 mm and about 10 mm, at times between about 2 mm and about 5 mm (in-plane resolution).

As noted herein, compounds of the invention and/or metabolites thereof are suitable for imaging and subsequent diagnosis. Diagnosis is required for the identification of specific subjects (sub-population) suffering from a specific disorder or condition.

In the context of the present disclosure, when referring to diagnosis and specifically to MRS and/or MRI diagnosis it should be understood to encompass a medical imaging technique used in radiology to provide spectra or to form pictures (images) of the anatomy and the physiological processes of the body in both health and disease.

As described herein, the 2DG-labeled compound and/or a metabolite thereof may be for use in the diagnosis of a condition of a disease.

In some embodiments, the method of the invention is utilized for determining a site of a disease or condition and/or for distinguishing between healthy and abnormal tissues or organs. In some embodiments, the method is used for distinguishing or differentiating between malignant and benign tumors.

In a further aspect, the invention provides a method for diagnosis of a disease or condition in a subject, the method comprising administering to the subject a diagnostically effective amount of a compound according to the invention and monitoring the subject or a body region of the subject to thereby identify body regions susceptible of having a disease or a condition. In some examples, the body regions susceptible of having a disease or a condition are associated with accumulation of a metabolite as described herein.

In another aspect, the invention provides a method for diagnosis of a disease or condition in a subject, the method comprising administering to the subject a diagnostically effective amount of a compound according to the invention and monitoring the subject or a body region of the subject to thereby identify body regions in which the compound or a metabolite thereof are accumulated.

In another aspect the invention provides a method for diagnosing a disease or condition or disease in a subject, said method comprising: administrating to the subject a diagnostically effective amount of a labeled compound and monitoring the compound or a metabolite thereof, thereby diagnosing the condition or disease in the subject.

In another aspect the invention provides a method for diagnosis of a disease or condition in a subject, the method comprising (i) administering to the subject a diagnostically effective amount of at least one 2DG compound and (ii) imaging and/or acquiring a spectrum of the subject or a body region of the subject to thereby identify body regions in which the compound, or any metabolite thereof has been localized, wherein the 2DG compound is deuterium-labeled 2DG.

The term “diagnostically effective amount” refers to the quantity of a substance (e.g. a 2DG compound as described herein) that is sufficient to produce a measurable effect or result for diagnostic purposes, without causing harm or toxicity. It is the dose needed to achieve the desired outcome, such as detecting a disease or condition, while ensuring patient safety.

The term “diagnosing a condition or disease” is meant to encompass any process of investigating, identifying, recognizing, assessing a condition, disease, or disorder of the mammalian body, including all tissues and structures in the body (for example, a tissue or blood vessels). A diagnosis according to the present disclosure using a compound described herein includes, but is not limited to objective quantitative diagnosis of a condition or disease, prognosis of a condition or disease, genetic predisposition of a subject to have a condition or disease, efficacy of treatment of a therapeutic agent administered to a subject (either continually or intermittently), quantification of neuronal function, diagnosis and evaluation from the fields of oncology, neurology, psychiatry, cardiology, vascular, infection and inflammation of a therapeutic agent activity, determination of drug efficacy, characterization of masses, tumors, cysts, blood vessel abnormalities, and internal organ function; quantification of brain, kidney, liver, and other organs' metabolic function; examination of the action, response or progress of therapy (involving medicinal and non-medicinal treatment) aimed at alleviating or curing at least one of oncology, neurology, psychiatry, cardiology, vascular, infection and inflammation diseases and disorders.

In some embodiments, the methods of the invention comprise a step of detecting a signal prior to administration of the compound. This may be possible by exposing the subject to a magnetic field using MR to obtain a detectable signal. In some embodiments, prior to administration of the compounds of the invention, at least one anatomical ¹H image may be acquired.

In some embodiments, prior to administration of the compounds of the invention, suspected regions of having a disease or condition may be recorded using similar conditions to the conditions at which the compound is acquired to obtain base-line information.

In some embodiments, the method comprising prior to the administration step, acquiring at least one ²H spectrum and/or at least one image from the subject's body or any one or more regions thereof. Monitoring the subject or any subject's region may be subjected to signal analysis of the MR spectrum or spectra and/or image processing of the acquired image(s). The results of the spectrum and/or image is indicative for the methods described herein.

In some embodiments, the method comprising comparing at least one parameter obtained from the at least one ²H MR spectrum and/or at least one ²H MR image to at least one parameter obtained from the at least one ²H MR spectrum and/or at least one ²H MR image in the same subject at an earlier point in time, wherein the comparison permits diagnosis of the disease.

In some embodiments, the method comprising comparing at least one parameter obtained spectrum and/or image analysis to at least one parameter obtained from spectrum and/or image analysis in the same subject obtained at an earlier point in time, wherein the comparison permits diagnosis of the disease. The earlier time point may be for example, prior to compound administration.

The compound, compositions or kits used according to the invention may be utilized for imaging a region or organ of a subject's body after or during treatment or otherwise state of a disease, it may be further utilized in determining severity of the disease, for, e.g., enabling determination of treatment effectiveness and continued treatment.

Therefore, in accordance with some aspects, it is provided a method for monitoring a disease state in a subject.

Monitoring a disease state as used herein refers to a process of tracking the progression of a disease or condition in a subject.

In such a method, the subject is administered the compound of the present disclosure, the subject's body or any one or more regions thereof is imaged, to obtain at least one imaging parameter indicative of the disease or disorder state, and comparing said at least one imaging parameter to at least one parameter obtained from said subject at an earlier point in time or upon identification of, e.g., at least one symptom associated with said disease or disorder, wherein the comparison permits determining the progression of the disease or disorder state.

Effective monitoring, made possible by utilization of a compound of the invention, involves obtaining multiple parameters indicative of a disease state and progression at various points in time, prior to, during or after commencement of treatment, and comparing the collected data to determine any one therapeutic parameter. The monitoring may be conducted over a period of time, for example every few days or weeks, once a week, once a month, at the onset of treatment and at any time thereafter, etc.

In a further aspect, the invention provides a method for determining the severity of a disease or disorder in a subject, the method comprising administering to said subject a compound according to the invention, imaging the subject's body or region thereof to obtain at least one imaging parameter (e.g., indicative of the state of the disease or disorder), and comparing said at least one imaging parameter to at least one parameter obtained from said subject at the onset of treatment or prior to treatment commencement, wherein the comparison permits determining the severity of the disease or disorder in the subject.

In another aspect, the invention provides a method for determining the effectiveness of a therapeutic treatment of a disease or disorder in a subject, the method comprising (i) administering to the subject a deuterium-labeled 2DG compound; (ii) acquiring at least one ²H MR image and/or ²H MR spectrum of the subject's body or region thereof to thereby obtain at least one imaging parameter; and (iii) comparing said at least one imaging parameter to at least one parameter obtained from said subject at the onset of treatment or prior to treatment commencement; thereby determining the effectiveness of the therapeutic treatment of the disease or disorder in the subject. In some embodiments, the compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₅]2-deoxy-D-glucose or a combination thereof, (c) [²H₄]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), Formula (XX), Formula (XXI), or a pharmaceutically acceptable salt, solvate, hydrate, any isomer thereof, any metabolite thereof or physiologically functional derivative thereof, or (f) a combination thereof.

In another aspect the invention provides a method for determining the effectiveness of a therapeutic treatment of a disease or disorder in a subject, the method comprising administering to said subject a compound according to the invention, imaging the subject's body or region thereof to obtain at least one imaging parameter (e.g., indicative of the state of the disease or disorder), and comparing said at least one imaging parameter to at least one parameter obtained from said subject at the onset of treatment or prior to treatment commencement, wherein the comparison permits determining the effectiveness of the therapeutic treatment of the disease or disorder in the subject.

The determination of the effectiveness of treatment may be achieved at the end of treatment or at any point in time during the treatment period. Generally, and depending on the disease and disease state, the effectiveness is indicated by any one or more changes in the disease state or any symptom associated therewith, such as decreased proliferation.

In some embodiments, the methods of the invention are used for evaluating the effectiveness of drug treatment in cancer treatment, for example, in evaluating the ability of a drug to reduce the size of a tumor or to prevent the tumor from growing, wherein the method comprises imaging the tumor with a compound according to the invention, as disclosed herein, and measuring the size of the tumor; administering the drug to the subject to affect at least one of reduction in the size of the tumor and prevention of growth of the tumor; re-imaging the tumor with the same or different compound and measuring the size of the tumor, and comparing the size of tumor after administration of the drug to the size of the tumor prior to administration of the drug. As compounds of the invention are not intended nor suitable for therapeutic use, the “drug” used for treatment is a material different from any compound used for diagnosis and accordance with the invention.

In some examples, the disease or condition is selected from oncology, neurology, psychiatry, cardiology, vascular, infection and inflammation.

Given the observed increased glycolysis in certain pathologies, for example, proliferative disease, the labeled 2DG described herein may be applicable in the diagnosis of such pathologies.

In some embodiments, the disease or condition is associated with increased glucose metabolism. There are various diseases that are associated with increased glucose metabolism and hence can be monitored using the compounds of the present disclosure and/or metabolites thereof as described herein.

In some examples, the disease or condition associated with increased glucose metabolism is a proliferative disease.

A proliferative disorder, diagnosed by utilizing compounds of the invention, is a disorder displaying cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper-proliferative disease” or “hyper-proliferative disorder.” It should be noted that the term “proliferative disorder”, “cancer”, “tumor” and “malignancy” all relate equivalently to a hyperplasia of a tissue or organ.

Non-limiting examples of cancers include blastoma, carcinoma, lymphoma, leukemia, sarcoma, mesothelioma, glioma, germinoma, choriocarcinoma, melanoma, glioblastoma, lymphoid malignancies, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments, the disease or condition is breast cancer. In some embodiments, the disease or condition is hepatocellular carcinoma. In some embodiments, the disease or condition is a metastatic tumour in the lungs.

In some embodiments, the compound and/or metabolite described herein are applicable for use in a method of distinguishing between healthy and abnormal tissues or organs and/or distinguishing or differentiating between malignant and benign tumors. As noted herein, without being bound by theory, it was suggested that high accumulation of the metabolite described herein indicate abnormal tissues or organs and/or malignant tumors (tissue).

In some examples, the disease or condition associated with increased glucose metabolism is an inflammatory disease.

An inflammatory disorder, diagnosed by utilizing compounds of the invention, is a disorder encompassing any immune response. The inflammatory disorder may be an infectious or a non-infectious disorder. Non-infectious inflammatory disorders are any disorder which the activation of macrophages or activated macrophages play a role such as auto-immune disorders and inflammatory disorders which are not infection related, i.e. non-pathogenic, caused by other than an infectious agent (e.g. auto-antigen, hypersensitivity, wound). Not limiting examples include inflammatory diseases of the gastrointestinal tract such as Crohn's disease, inflammatory bowel disease, gastritis, colitis, ulcerative colitis, colon irritable, gastric ulcer and duodenal ulcer, inflammatory diseases of the skin such as psoriasis, inflammatory diseases of the respiratory system such as asthma, allergic rhinitis or chronic obstructive pulmonary disease (COPD), pulmonary fibrosis, sarcoidosis, inflammatory diseases of the musculoskeletal system such as rheumatoid arthritis, osteomyelitis, osteoporosis, or neuritis, systemic sclerosis, inflammatory diseases of the kidneys such as glomerulonephritis, renal ischemia, or renal inflammation; inflammatory diseases of the nervous system such as multiple sclerosis, Alzheimer's disease and HlV-1-associated dementia; autoimmune diseases such as diabetes, type 1 and 2 diabetes mellitus and graft versus host reaction; infectious disease such as nephritis, sepsis, septic shock, endotoxic shock, adult respiratory distress syndrome; inflammatory conditions of the cardiovascular system, such as myocardial infarction, myocarditis, atherosclerosis, hypertensive cardiomyopathy, atheroma, intimal hyperplasia or restenosis or autoimmune disorders such as Multiple Sclerosis (MS), inflammatory arthritis, rheumatoid arthritis (RA).

In some examples, the disease or condition associated with increased glucose metabolism is a cardiovascular disease. In some examples, the cardiovascular disease or condition is coronary artery disease or stroke. In some examples, the cardiovascular disease is ischemic heart disease (coronary artery disease) or stroke. In some examples, the disease or condition associated with increased glucose metabolism is a vascular disease. In some examples, the vascular disease is ischemic cerebrovascular disease.

In some examples, the disease or condition associated with increased glucose metabolism is a neurological disease. In some examples, the neurological disease is a neurodegenerative disease. In some examples, the neurodegenerative disease is Alzheimer disease. In some examples, the neurological disease is epilepsy.

In some examples, the disease or condition associated with decreased glucose metabolism is a neurological disease. In some examples, the neurological disease is a neurodegenerative disease. In some examples, the neurodegenerative disease is stroke.

In some examples, the disease or condition associated with decreased glucose metabolism is a systemic disease. In some examples, the diseases or condition cause ischemia in bodily organs or the brain. In some examples, the systemic disease or condition is diabetes or metabolic syndrome. In some examples, the ischemic condition is in the legs.

The methods of the present discourse provide a method for treating a disease or condition in a subject. The method comprises: First (a), diagnosis of a disease or condition in a subject according to the method of the invention described above.

The second step (b) involves administrating to the subject a treatment regimen based on said disease or condition thereby treating the subject.

Hence, the present disclosure provides in accordance with some aspects, a method for treating, preventing, inhibiting, reducing, eliminating, protecting or delaying the onset of at least one condition in a subject in need thereof, the method comprises diagnosing a disease or condition in a subject according to the method of the invention described above and administration to the subject of a treatment regimen based on said disease or condition thereby treating the subject.

As used herein, “disease”, “disorder”, “condition” and the like, as they relate to a subject's health, are used interchangeably and have meanings ascribed to each and all of such terms.

It should be noted that for the method of treatment and prevention provided in the present invention, may use therapeutic effective amount, or dosage, dependent on severity and the disease state to be treated, with the course of treatment lasting from several days to several months, or until a cure is affected or a diminution of the disease state is achieved. Optimal dosing schedules can be calculated from measurements of drug accumulation in the body of the patient.

The term subject as used herein refers to human and non-human subjects.

Specifically, the subject in accordance with the present disclosure is suspected to or suffering from a pathological disorder or a related condition.

It is understood that the interchangeably used term “associated” or “related”, when referring to pathologies herein, mean diseases, disorders, conditions, or any pathologies which at least one of share causalities, co-exist at a higher than coincidental frequency, or where at least one disease, disorder condition or pathology causes the second disease, disorder, condition or pathology.

As noted herein, the present disclosure relates to pharmaceutically acceptable salt, solvate, hydrate, stereoisomer, metabolite, or physiologically functional derivative thereof.

The term “pharmaceutically acceptable salt” refers to salts derived from organic and inorganic acids of the SMC described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of a compound described herein.

The term “solvate” refers to an aggregate of a compound with one or more solvent molecules, such as hydrate, alcoholate (aggregate or adduct with alcohol), and the like.

The term “hydrate” refers to a compound formed by the addition of water. The hydrates may be obtained by any known method in the art by dissolving the compounds in water and recrystallizing them to incorporate water into the crystalline structure.

The term “tautomer” refers to a compound that exists in equilibrium with another compound having the same molecular formula but differing in the arrangement of atoms and the position of hydrogen atoms and/or double bonds. Tautomers interconvert via a chemical process such as proton transfer or the shifting of bonds, typically in response to changes in environmental conditions such as pH or solvent polarity. Examples include the equilibrium between the linear and cyclic forms of a monosaccharide or between keto and enol forms of a compound

The term “isomer” refers to a compound that shares the same molecular formula with another compound but differs in the arrangement of its atoms or the spatial orientation of its functional groups. Isomers can include structural isomers (differing in the connectivity of atoms) and stereoisomers (differing in the spatial arrangement of atoms, such as enantiomers or diastereomers). Examples include D-glucose and L-glucose (enantiomers) or α-D-glucose and β-D-glucose (anomers).”

The term “metabolite” or “pharmaceutically acceptable metabolite” as used herein refers to a compound that is formed under physiological conditions to of degrading and eliminating the compounds. Oxidative metabolite may an example. Specifically, as described herein, the metabolite may be a phosphorylated compound.

The term “physiologically functional derivative” used herein relates to any physiologically acceptable derivative of a compound as described herein. The physiologically functional derivatives also include prodrugs of the compounds of the invention.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to +10%.

It should be noted that various embodiments of this invention may be presented in a range format. The description of a range should be considered to have specifically disclosed all the possible sub ranges as well as individual numerical values within that range inclusive the end points values. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It should be noted that the term “at least one” refers to one or more of the following and encompasses any one of 1, 2, 3, or more of the specified information.

It is to be understood that the terminology used herein is used for the purpose of describing particular embodiments only and not intended to be limiting since the scope of the present invention will be limited only by the appended claims and equivalents thereof.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It should be noted that the various embodiments and examples detailed herein in connection with various aspects of the invention may be applicable to one or more aspects disclosed herein. It should be further noted that any embodiment described herein may be applied separately or in various combinations. Various embodiments and aspects of the present invention as delineated hereinabove and as claimed in the claims section below find experimental support in the following examples. The phrases “in another embodiment” or any refence made to embodiment as used herein do not necessarily refer to different embodiment, although it may. Thus, various embodiments of the invention can be combined (from the same or from different aspects) without departing from the scope of the invention.

### SOME NON-LIMITING EXAMPLES

### Material and Methods

### Synthesis of deutero-2-deoxy-D-glucose

Isotopically substituted 2DG compounds can be synthesized by any method known in the art. For example, per-O-acetylated D-glucal may be used as the starting material and being deacetylated and benzylated. Further, D2O may be added in the presence of DBr in tetrahydrofuran.

### Example 1: Determinations of Deuterons T₁ in Sugar Molecules

Table 1 describes the difference in T₁ of deuterons in water (HDO, natural abundance) and in sites labelled with deuterons in various molecules that were determined using an Inversion Recovery technique. As can be seen from Table 1, deuterons that are directly bound to carbons in a ring (glucose or tyrosine) or aliphatic deuterons near the ring, show very short T₁s of 40-60 ms, while methylene positions (choline) show longer T₁ that is of the order of HDO's T₁ (ca. 400 ms). It is also demonstrated that binding to ¹³ C or ¹²C nuclei does not affect the deuteron's T₁ in that position, which further suggests that motion restriction is the dominant mechanism for T₁ shortening of the sugar deuterons. PGP-47 TI

TABLE 1
T₁ relaxation times of deuterons directly bound
to carbons in an aqueous saline solution ^{a}

| | | HDO | D site |
|---|---|---|---|
| Compound | D site/position | T₁ (ms) | T₁ (ms) |
| [¹³C₆, D₇]glucose ^{b} | Aliphatic + ring | 279 | 56 |
| [6,6-D₂]D-glucose | aliphatic | 420 | 53 ^{c} |
| [¹³C₉, D₇, ¹⁵N]tyrosine | aromatic | 410 | 45 |
| | aliphatic | | 45 |
| [D₇]tyrosine | aromatic | 420 | 43 |
| | aliphatic | | 48 |
| [2-¹³C,1,1,2,2-D₄]choline | 1 (aliphatic) | 437 | 422 |
| chloride | 2 (aliphatic) | | 404 |
| [D₁₃]choline bromide | 1 (aliphatic) | 422 | 389 |
| | 2 (aliphatic) | | 396 |
| | 3 (aliphatic) | | 382 |

^{a} All measurements were carried out at 5.8T in medical grade saline solution, at 16-17 °C, except the measurement marked
^{b} which was performed at 20 °C.

### Example 2: Recording of [6,6-D₂]D-Glucose Uptake and Metabolism in Brain Slices During Stress

### Materials and Methods

### Chemicals

[6,6-D₂]D-glucose was obtained from Cambridge Isotope Laboratories (Tewksbury, MA, USA). NaCl, KCl, D-glucose, NaHCO₃, MgSO₄, NaH₂PO₄, N-methyl-D-glucamine (NMDG), HEPES, pyruvic acid, ascorbic acid, thiourea, and CaCl₂ were purchased from Sigma-Aldrich, (Rehovot, Israel). Isoflurane was obtained from the Institutional Authority for Biological and Biomedical Models of the Hebrew University (Jerusalem, Israel).

### Animals

Male Sprague-Dawley rats (n=4, 70-100 g) were obtained from the Hebrew University Authority of Biological and Biomedical Models. The joint ethics committee (IACUC) of the Hebrew University and Hadassah Medical Center approved the study protocol for animal welfare. The Hebrew University is an AAALAC International accredited institute. Care was taken to minimize the animals' pain and discomfort. Animals were housed in the animal facilities 3-5 days after delivery for acclimatization and fed ad libitum. On the experimental day, animals were transferred to the laboratory and anesthetized within one hour of arrival.

### Solutions

To improve the health of acute brain slices obtained from mature adult rats, we modified a slice preparation technique optimized for in vitro electrophysiology in mature adult rat brain slices. In brief, slice health is improved by a so-called “protective recovery” method. NMDG is an extracellular replacer of sodium ions that prevents sodium ions and water influx into cells, the primary insult causing cell death following brain slicing. Ting et al. showed that the presence of NMDG during a 12-minute recovery phase after slicing dramatically improved the health of acute slices from mature adult rats in electrophysiological recordings. Furthermore, incubation in HEPES-buffered artificial cerebrospinal fluid (aCSF) improved slice health. The following solutions were prepared in the current study, and their use is described in the sections below.

### Solution 1—NMDG-aCSF

NMDG-aCSF was used for transcardial perfusion, tissue slicing, and slice recovery. This medium contained 93 mM NMDG, 2.5 mM KCl, 1.2 mM NaH₂PO₄, 26 mM NaHCO₃, 20 mM HEPES, 25 mM D-glucose, 10 mM MgSO₄, 0.5 mM CaCl₂, 5 mM ascorbic acid, 2 mM thiourea, and 3 mM pyruvic acid in double distilled water.

Solution 2—HEPES-holding aCSF

HEPES-holding aCSF was used to incubate slices following the surgical procedure. This medium contained 84 mM NaCl, 2.5 mM KCl, 1.2 mM NaH₂PO₄, 30 mM NaHCO₃, 20 mM HEPES, 25 mM Glc (D-glucose, non-labeled), 2 mM MgSO₄, 2 mM CaCl₂, 5 mM ascorbic acid, 2 mM thiourea, and 3 mM pyruvic acid in double distilled water.

Solution 3—Perfusion aCSF

Perfusion aCSF was used for perfusion in the NMR spectrometer. This medium contained 115 mM NaCl, 2.5 mM KCl, 1.2 mM NaH₂PO₄, 24 mM NaHCO₃, 5 mM HEPES, 10 mM Glc (to be replaced with 10 mM D-Glc), 2 mM MgSO₄, and 2 mM CaCl₂ in double distilled water.

All three solutions (1-3) were bubbled with 95%/5% O₂/CO₂ for at least one hour before use and titrated to a pH of 7.35-7.45 using HCl or NaOH. All concentrations were optimized for a calculated osmolarity of 310±15 mOsm.

### Surgery and Brain Slices

Brain slices were extracted and maintained using the above solutions as previously described. Briefly, the rats were anesthetized using a gas anesthesia system (Somnosuite, Kent Scientific, Torrington, CT, USA). Induction was performed in a chamber with 3.5% isoflurane in room air with a flow rate of 440 mL/min. Following 4-7 min of induction, anesthesia was maintained with 3.1-3.2% isoflurane at the same flow rate. Upon obtaining a negative pedal pain reflex, surgery was initiated. First, the diaphragm was exposed and cut, and the rat was transcardially perfused with 30 mL of ice-cold NMDG-aCSF (Solution 1). The animals were then sacrificed by decapitation, and the brain was rapidly removed and placed in ice-cold NMDG-aCSF (Solution 1). The cerebrum was then cut into four parts (first, a sagittal cut along the hemispheric cleft and then a sagittal cut in the middle of each hemisphere). From each part, 350 μm slices were prepared using a McIlwain tissue chopper (The Mickle Laboratory Engineering Company Ltd., Surrey, UK). The process of brain extraction, from decapitation till the brain was in ice-cold NMDG-aCSF, took under 2 min, and the slicing procedure was done in less than 10 min. After cutting, the slices were transferred to warm, 32-34 °C, NMDG-aCSF (Solution 1) for 12 min for protective recovery. Then, the slices were transferred to HEPES-holding aCSF (Solution 2) at ~32 °C for 15-40 min incubation before transfer to the NMR spectrometer, where the slices were perfused with the aCSF of Solution 3.

Calculation of Deuterated Compounds' Concentrations from Deuterium NAMR Spectra

The natural abundance of HDO in water was used as an internal reference to calculate the concentration of the deuterated metabolites in the sample in each spectrum. To reduce the variability of the data that may result from variations in this internal standard's signal, the HDO signal was first averaged over ten spectra. Then, each HDO signal in all the spectra was converted to concentration units in the following way.

[HDO]=S_{HDO}*17.16/S_{AV_HDO}, where S_{HDO} is the individual HDO signal in each spectrum, [HDO] is the concentration equivalent of each S_{HDO}, and S_{AV_HDO} is the averaged HDO signal from 10 spectra at the beginning of the experiment (during perfusion with D-Glc). As the natural abundance of deuterium is 0.0156% and the concentration of protons in water is 110 M, the theoretical concentration of HDO is 17.16 mM (0.0156%*110 M).

For D-Glc and D-Lac, there are two deuterium nuclei per molecule. Therefore, their concentrations ([D-Glc] and [D-Lac], respectively) were calculated as follows.

[D-Glc]=S_{D-Glc}*(17.16/S_{AV_HDO})/2, where S_{D-Glc} is the individual signal of D-Glc.

and

[D-Lac]=S_{D-Lac}*(17.16/S_{AV_HDO})/2, where S_{D-Lac} is the individual signal of D-Lac.

Note that the total concentration of lactate (deuterium-labeled and non-labeled) produced from D-Glc is twice that of D-Lac.

### Perfusion of Brain Slices in the NMR Spectrometer and Experimental Workflow

The perfusion system was operated as described previously at 4 ml/min. D-Glc was introduced to the slices by addition to the external aCSF reservoir. To allow the utilization of a minimal amount of the labeled compound, 70 mL of aCSF was used for the perfusion of the slices with D-Glc.

### Spectral Analysis

Spectral processing and intensity integrals were calculated using MNova (Mestrelab Research, Santiago de Compostela, Spain).

### Statistical Analysis

Statistical analysis was performed in Excel (Microsoft Office, Rānana, Israel).

### Results

Brain slices can be prepared by any method known in the art. Further details about this system can be found at Grieb et al.

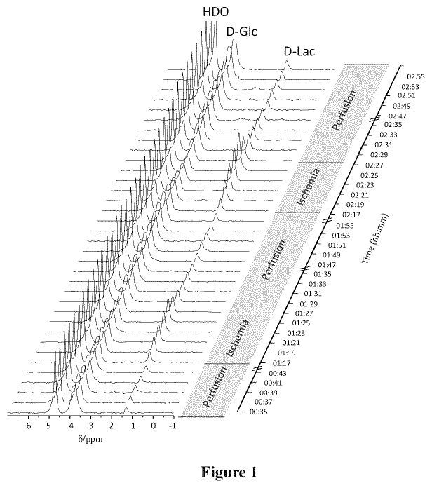

A time course of glucose metabolism in perfused and ischemic brain is shown in FIG. 1.

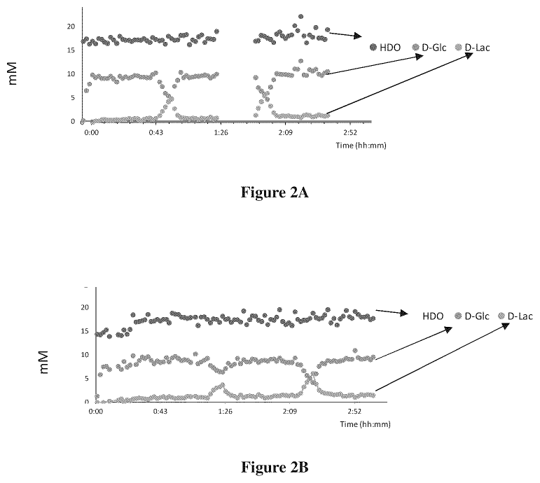

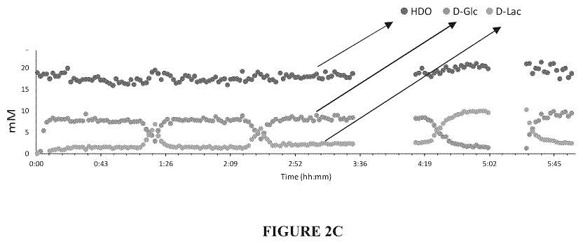

Individual and averaged D-Glc and D-Lac levels in each experiment and within the various experimental sections are provided in Table 2 and FIG. 2.

TABLE 2
A summary of individual data used for statistical analysis is in
the results section.

| | D-Glc or | | | | Average ± | |
|---|---|---|---|---|---|---|
| | D-Lac, | | | | Standard | |
| | timing, and | Animal | Animal | Animal | Deviation | P of |
| Group | condition | 1 (mM) | 2 (mM) | 3 (mM) | (mM) ^{a} | t-test |
| A | D-Glc | 9.3 | 8.8 | 7.6 | 8.6 ± 0.7 | na |
| | before 1^{st} | | | | | |
| | ischemia | | | | | |
| B | Minimum | 4.7 | 6.4 | 2.8 | 4.6 ± 1.5 | 0.035 |
| | D-Glc | | | | | A-B |
| | during 1^{st} | | | | | |
| | ischemia | | | | | |
| B′ | Maximal | 4.6 | 2.4 | 4.8 | 3.9 ± 1.3 | na |
| | difference | | | | | |
| | of D-Glc | | | | | |
| | before and | | | | | |
| | during 1^{st} | | | | | |
| | ischemia | | | | | |
| C | D-Glc in | 9.5 | 8.6 | 7.9 | 8.7 ± 0.7 | na |
| | between | | | | | |
| | ischemic | | | | | |
| | conditions | | | | | |
| D | Minimum | 4.6 | 4.2 | 3.4 | 4.1 ± 0.5 | P = |
| | D-Glc | | | | | 0.6 |
| | during 2^{nd} | | | | | B-D |
| | ischemia | | | | | P = |
| | | | | | | 0.001 |
| | | | | | | A-D |
| D′ | Maximal | 4.9 | 4.4 | 4.5 | 4.6 ± 0.3 | na |
| | difference | | | | | |
| | of D-Glc | | | | | |
| | before and | | | | | |
| | during 2^{nd} | | | | | |
| | ischemia | | | | | |
| E | D-Glc | 10.8 | 8.8 | 8.2 | 9.3 ± 1.1 | A-E |
| | after 2^{nd} | | | | | 0.2 |
| | ischemic | | | | | |
| | condition | | | | | |
| F | D-Lac | 0.4 | 1.02 | 1.4 | 0.9 ± 0.4 | na |
| | before 1^{st} | | | | | |
| | ischemia | | | | | |
| G | Maximal | 4.8 | 3.7 | 6.8 | 5.1 ± 1.3 | F-G |
| | D-Lac | | | | | 0.035 |
| | during 1^{st} | | | | | |
| | ischemia | | | | | |
| H | D-Lac in | 0.67 | 1.29 | 1.41 | 1.1 ± 0.3 | F-H |
| | between | | | | | 0.169 |
| | ischemic | | | | | |
| | conditions | | | | | |
| I | Maximal | 5.85 | 6.07 | 5.80 | 5.91 ± 0.12 | I-F, |
| | D-Lac | | | | | 0.004 |
| | during 2^{nd} | | | | | I-G |
| | ischemia | | | | | 0.502 |
| J | D-Lac | 1.08 | 1.50 | 2.16 | 1.6 ± 0.4 | J-H, |
| | after 2^{nd} | | | | | 0.101 |
| | ischemic | | | | | J-F |
| | condition | | | | | 0.017 |

| | D-Glc or | | | | |
|---|---|---|---|---|---|
| | D-Lac, | | | | |
| | timing, and | Animal | Animal | Animal | Error for single |
| Group | condition | 1 (mM) | 2 (mM) | 3 (mM) | measurements |
| K | D-Glc in | na | na | 8.3 | 0.4 ^{b} |
| | between | | | | |
| | 2^{nd} and 3^{rd} | | | | |
| | ischemic | | | | |
| | conditions | | | | |
| L | Minimum | na | na | 1.2 | 0.2 ^{c} |
| | D-Glc | | | | |
| | during 3r^{d} | | | | |
| | ischemia | | | | |
| L′ | Maximal | na | na | 7.1 | 1.1 ^{c} |
| | difference | | | | |
| | of D-Glc | | | | |
| | before and | | | | |
| | during 3^{rd} | | | | |
| | ischemia | | | | |
| M | D-Glc | na | na | 9.1 | 0.5 ^{b} |
| | after 3^{rd} | | | | |
| | ischemic | | | | |
| | condition | | | | |
| N | Maximal | na | na | 10.2 | 1.5 ^{c} |
| | D-Lac | | | | |
| | during 3^{rd} | | | | |
| | ischemia | | | | |
| O | D-Lac | na | na | 2.7 | 0.3 ^{b} |
| | after 3^{rd} | | | | |
| | ischemia | | | | |

na, not applicable
^{a} average, and standard deviation are for the three values for the three animals.
^{b} the error is the standard deviation of the ten points used to determine this value.
^{c} the error was taken as 15% of the single value of the single point in the time course.

The HDO, D-Glc, and D-Lac signals are consistently observed and allow the calculation of the concentration changes during the ischemic conditions. The time scale is in hh:mm (h, hour; m, min). The time of addition of D-Glc to the external perfusion medium was taken as the starting time (00:00). The durations of continuous perfusion and flow arrest (1^{st} and 2^{nd} ischemic conditions) are marked. HDO, the natural abundance of deuterium in water.

Upon administration of D-Glc to the external reservoir of the perfusion medium, its signal (in the NMR tube) reached a plateau within 6 min (8.6±0.7 mM). The D-Lac signal was observed within 8 min of the D-Glc administration, increased, and reached a plateau of 0.9±0.4 mM (n=3) within 12-14 min.

The perfusion was arrested for 10 min (1^{st} ischemic condition) at 49-76 min (n=3) from the administration of D-Glc. During this period, the signal of D-Lac increased gradually to a maximal level of 5.1±1.3 mM (n=3), and the signal of D-Glc decreased gradually to a minimal level of 4.6±1.5 mM (n=3, two-tailed, paired t-test, before and at the end of the 1^{st} ischemic condition, P=0.035). After the 1^{st} ischemic condition, the perfusion was resumed. Upon reperfusion, within about 8 min, the signal of D-Glc returned to the baseline level of 8.7±0.7 mM (n=3), and the signal of D-Lac returned to baseline at a level of 1.1±0.3 mM (two-tailed, paired t-test before and after ischemia, P=0.17, no difference). Continuous perfusion followed the 1^{st} ischemic condition for 53 70 min, and then the perfusion was again arrested for 10 min (2^{nd} ischemic condition).

During the 2^{nd} ischemic condition, the D-Lac concentration gradually increased to a similar maximal level of 5.91±0.12 mM (n=3, two-tailed, paired t-test, 1^{st} and 2^{nd} ischemic conditions, P=0.50, no difference). The signal of D-Glc decreased to 4.1±0.5 mM, as in the 1^{st} ischemic condition (n=3, two-tailed, paired t-test, 1^{st} and 2^{nd} ischemic conditions, P=0.56, no difference, and two-tailed, paired t-test, before and at the 2^{nd} ischemic condition, P=0.001). Further to the 2^{nd} ischemic condition, the perfusion was resumed, and again, within about 8 min, the D-Glc signal returned to the baseline level of 9.3±1.1 mM (two-tailed, paired t-tests before the 1^{st} ischemic condition and after the 2^{nd} ischemic condition, P=0.2). The D-Lac signal further to reperfusion after the 2^{nd} ischemic condition was similar to its level before the 1^{st} ischemic condition and between the two ischemic conditions, indicating that D-Lac did not accumulate within the cells due to the ischemic conditions (P=0.4, ANOVA with replication).

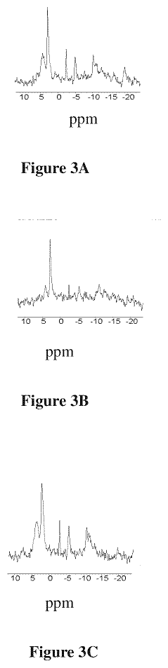

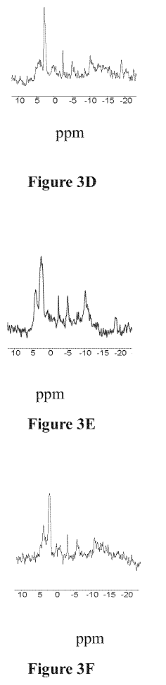

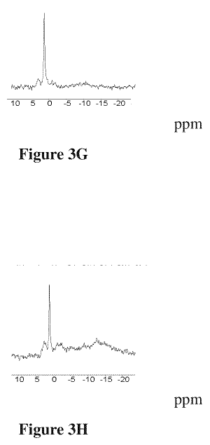

On both ischemic conditions, the increase in D-Lac concentration was the same as the maximal decrease in the D-Glc concentration (5.5±1.1 mM and 4.3±0.9 mM, respectively, n=6, P=0.2, no difference, two-factor ANOVA with replication). This suggested that the D-Glc available to the brain slices during the ischemic conditions was fully converted to D-Lac (and, by inference, also to the same amount of non-labeled lactate). ³¹P NMR spectra of the slices recorded before the 1^{st} ischemic condition and after the 2^{nd} ischemic condition confirmed the viability of the slices and the consistent presence of ATP and PCr in the slices. The individual spectra are shown in FIG. 3. Information about the timing of acquiring the individual spectra of FIG. 3 is provided in Table 3.

TABLE 3
Information about the timing of acquiring
the individual spectra of FIG. 3

| | Animal/ | |
|---|---|---|
| FIG. | Experimental day | Experimental section |
| 3A | 1 | Before 1^{st} ischemia |
| 3B | 1 | After 2^{nd} ischemia |
| 3C | 2 | Before 1^{st} ischemia |
| 3D | 2 | After 2^{nd} ischemia |
| 3E | 3 | Before 1^{st} ischemia |
| 3F | 3 | After 2^{nd} ischemia |
| 3G* | 3 | During 3^{rd} ischemia |
| 3H | 3 | After 3^{rd} ischemia |

*All spectra were acquired for 30 minutes except spectrum G, which was acquired for 15 minutes.

Analysis of ATP content in the slices, comparing the state before the 1^{st} ischemic condition and after the 2^{nd} ischemic condition, showed that during this time (about five h), it was decreased by 28±5% (n=3, average±standard deviation). This analysis was based on the γ-ATP signal, as described previously.

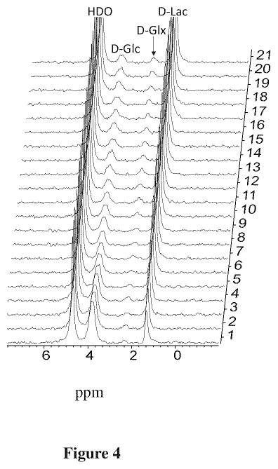

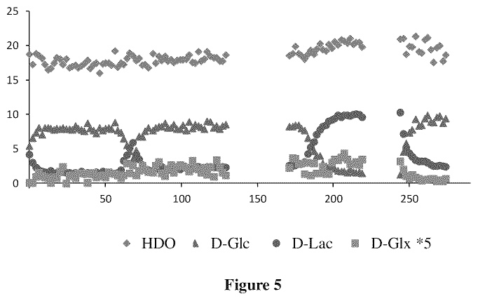

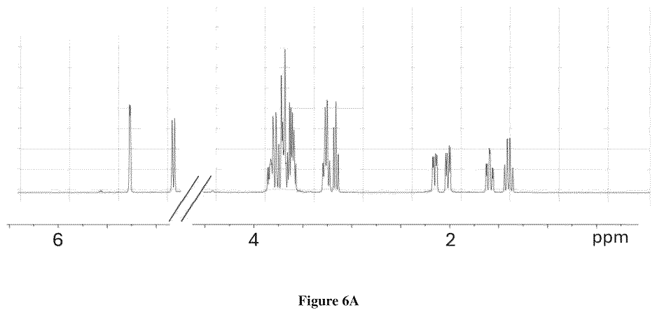

In one of these experiments, an additional 3^{rd} ischemic condition was tested, which was applied at 87 min after the 2^{nd} ischemic condition for 64 min. In this 3^{rd} ischemic condition, the D-Lac concentration increased to 10.2 mM, and the D-Glc concentration decreased to 1.2 mM. A ³¹P spectrum acquired towards the end of this ischemic condition showed almost no high-energy phosphate signals. However, 30 min further to reperfusion, some recovery was observed. Interestingly, in this experiment, D-Glx was sporadically observed during the 1^{st} ischemic condition and afterward. The level D-Glx increased towards the end of the long 3^{rd} ischemic condition (FIG. 4 and FIG. 5).

As can be seen in FIG. 4, in addition to the HDO, D-Glc, and D-Lac signals, which are consistently observed, the signal of D-Glx can also be observed. The data are shown at 2-minute intervals starting at the beginning of the third ischemic condition and before reperfusion. The top spectrum, marked spectrum 21, was acquired at the end of the third ischemic condition, 22 min after the spectrum marked 20, and after 62 min of ischemia

In a control experiment without perfusion arrests (no ischemic conditions), the level of D-Lac was constant throughout, at a level similar to the three ischemia experiments under continuous flow. In a spectrum acquired without brain slices from the medium used for perfusion of these slices (without perfusion arrest) for five h in the presence of D-Glc (acquired at the same perfusion flow), the D-Lac signal was found to be 2.2% of the level of D-Lac in the NMR tube containing the brain slices.

FIG. 5 shows the partial time course of the experiments, Breaks in D-NMR acquisitions are taken to record ³¹P spectra.

### Example 3: Synthesis of [6,6-D₂]2-Deoxy-D-Glucose

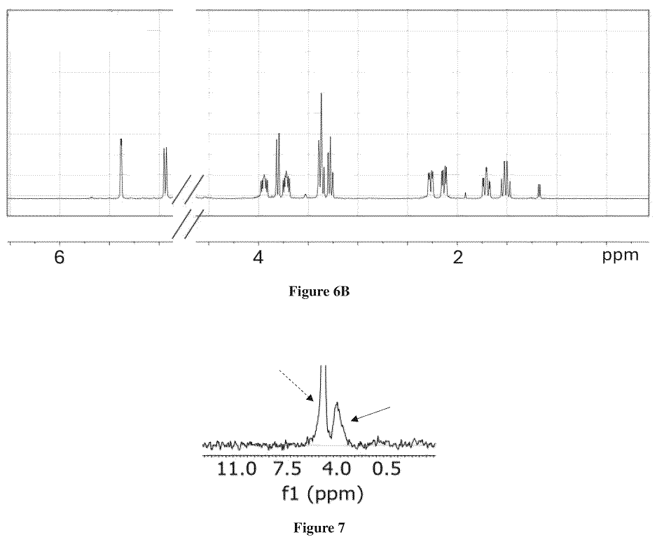

FIG. 6A is a ¹H NMR spectra of commercial 2-deoxyglucose-6,6-H₂ and FIG. 6B is an ¹H NMR spectra of 2-deoxyglucose-6,6-D2. The disappearance of the 1H signals (integration 2 protons) in the 3.8 ppm region indicates the successful replacement of two protons by two deuterium in position 6,6.

A normal mammalian brain as a model system will be used to estimate the rate of [D₈]2-deoxy-D-glucose uptake by active tissues. The system of rat brain slices perfused with artificial cerebrospinal fluid (aCSF) provides a suitable model system for this investigation. In addition, this system will be used for initial evaluation of [D₈]2-deoxy-D-glucose uptake in breast cancer, utilising breast cancer xenografts from triple-negative tumours.

### Example 4: Initial Assessment of Effective Dose

The effective dose for [D₈]2-deoxy-D-glucose is evaluated in vivo with suitable hardware and methodologies (RF coils/insert and pulse sequences).

As an initial dose, the amount of [D₈]2-deoxy-D-glucose that is injected intravenously can be calculated to be about 4.3 g per patient (ca. 60 mg/kg).

For injection in an isosmotic condition, a formulation of about 75 mL solution with a [D₈]2-deoxy-D-glucose concentration of 330 mM is injected.

### Example 5: Detecting and Characterizing Breast Lesions

Detecting and characterizing breast lesions in women with high risk for developing breast cancer is done using [D8]2-deoxy-D-glucose.

A woman with high-risk for developing breast cancer is scanned by MRI with contrast enhancement using the standard-of-care MRI protocol, as part of her annual examination.

Prior to gadolinium-based contrast agent injection for contrast-enhanced MRI, the women will be intravenously injected with [D8]2-deoxy-D-glucose at a dose of about 60 mg/kg.

Imaging of deuterium distribution by deuterium-MRI according to the method of the invention will depict the distribution of [D8]2-deoxy-D-glucose in the breast. This will indicate the anatomical location of metabolically active regions in the breast. Taken together with contrast-enhanced imaging results, anatomical MRI images, and the finding of other breast imaging modalities (ultrasound and mammography), this information will enable the breast radiologist to reach a highly sensitive and highly specific diagnosis of the lesion(s) that may be found in the breast.

The inventors have evidence that [D4]2-deoxy-D-glucose ([3,4,6,6-D₄]2-deoxy-D-glucose), when administered to female mice (0.5 g/kg intravenous) bearing breast cancer tumor, may be visualized using deuterium MRI over a wide range of doses and at multiple time points extending from 30 minutes to at least 48 hours after administration (data not shown). Under the acquisition conditions that were used, no signal for natural-abundance deuterium in water is observed in the mice (data not shown).

### Example 6: Detecting and Characterizing Stroke and Ischemia in the Brain

Detecting and characterizing stroke and ischemia in the brain is done using [D8]2-deoxy-D-glucose.

A person is scanned by MRI using the standard-of-care MRI protocol, as part of the standard-of-care established in a particular medical center.

Prior to the MRI, the patient will be intravenously injected with [D8]2-deoxy-D-glucose at a dose of about 60 mg/kg.

Imaging of deuterium distribution by deuterium-MRI in the brain according to the method of the invention will depict the distribution of [D8]2-deoxy-D-glucose in the brain. This will indicate the anatomical location of metabolically inactive regions in the brain. Taken together with anatomical MRI images, diffusion-weighted MRI and other MRI characteristics, this information will enable the radiologist and neuroradiologist to reach a highly sensitive and highly specific characterization of the condition and stratify treatment.

### Example 7: Detecting and Characterizing Ischemic Legs and Feet

Detecting and characterizing ischemia in the legs of diabetes patients is done using [D8]2-deoxy-D-glucose.

A patient is scanned by MRI with contrast enhancement using the standard-of-care MRI protocol, as part of the standard-of-care established in a particular medical center.

Prior to the MRI, the patient will be intravenously injected with [D8]2-deoxy-D-glucose at a dose of about 60 mg/kg.

Imaging deuterium distribution by deuterium-MRI in the legs and feet according to the method of the invention will depict the distribution of [D8]2-deoxy-D-glucose in the legs and feet. This will indicate the anatomical location of metabolically inactive regions in the legs and feet. Taken together with anatomical MRI images, diffusion-weighted MRI and other MRI characteristics, this information will enable the radiologist and the diabetes or metabolic specialist to reach a highly sensitive and highly specific characterization of the condition and stratify treatment.

### Example 8: Biodistribution Study of Deuterated 2-Deoxy-D-Glucose

### Materials and Methods

Mice and rats, males and females at equal numbers, were intravenously injected with a bolus of varying doses of deuterated 2-deoxy-D-glucose (2-DG, DDG, with varying numbers and positions of deuteration). DDGs were provided by CortecNet, France. The injection was performed under sedation in all rats (18) and in 12 of 18 mice, after which the animals woke up within 1 minute. At various times after the bolus injection, when the animals were awake, exhibited normal behavior, and showed no signs of stress, they were sacrificed with CO₂. Further to euthanasia, the brain, liver, kidneys, heart, and muscle were excised and immediately placed in liquid nitrogen. The frozen tissue samples were kept at −80 °C until further processing.

Extraction of the water-soluble metabolites and the lipids was carried out using the dual-phase extraction procedure as described previously [Katz-Brull R, et al. Differential routing of choline in implanted breast cancer and normal organs. Article. Magnetic Resonance in Medicine. July 2001; 46(1):31-38. doi:10.1002/mrm.1157]. Briefly, a frozen sample is placed in 10 mL of ice-cold methanol containing 0.4 mM phenyl phosphonic acid for at least 1 hour. The tissue is then homogenized in ice-cold methanol using a tissue homogenizer. Equal volumes of chloroform and water are then added to the sample tube, and the mixture is vortexed and vigorously shaken. The samples are allowed to stand at 4 °C for at least 24 hours for phase separation. The methanol-water phase is aspirated, treated with Chelex to remove divalent ions, and then lyophilized for at least 24 hours. When the samples are dry (as powder), they are redissolved in 0.5 mL of double-distilled water and transferred to 5-mm NMR tubes for scanning. The samples are scanned in a 9.4 T NMR spectrometer.

### Results

Altogether, 18 mice and 18 rats were injected with varying doses of three DDG types as described in Table 4. All animals showed no adverse effects. One mouse died in the first experiment due to inadvertent anesthesia overdose and showed no abnormality on pathology.

TABLE 4
Summary of DDG doses and durations
of circulation in awake rodents.

| Experi- | | Number | Dose | Duration | |
|---|---|---|---|---|---|
| mental | Animal | of | range | range | DDG |
| group | type | animals | (mg/kg*) | (min) | analogue |
| 1 | Mouse | 6 | 42-45 | 30-45 | [D₄]DDG - |
| 2 | Rat | 6 | 37-55 | 30-41 | [3,4,6,6-D₄]2- |
| | | | | | deoxy-D- |
| | | | | | glucose |
| 3 | Mouse | 2 | 87, 89 | 39, 45 | [D₂]DDG - |
| 4 | Rat | 2 | 43, 39 | 39, 66 | [6,6-D₂]2- |
| 5 | Mouse | 2 | 124, 135 | 40-45 | deoxy-D- |
| 6 | Rat | 2 | 86, 78 | 58, 75 | glucose |
| 7 | Mouse | 2 | 171, 184 | 41, 46 | |
| 8 | Rat | 2 | 118, 104 | 67, 76 | |
| 9 | Mouse | 6 | 170-194 | 19-78 | [D₈]DDG - |
| 10 | Rat | 6 | 182-248 | 33-71 | [1,2,2,3,4,5,6,6- |
| | | | | | D₈]2-deoxy- |
| | | | | | D-glucose |

*Non-labelled 2DG dose equivalent

Mice and rats showed no adverse effects to bolus intravenous injections of [D₂]DDG, [D₄]DDG, and [D₈]DDG at doses ranging from 37 mg/kg to 248 mg/kg. The animals were observed awake for up to 78 min after the injection. The injection bolus was administered in less than 1 s and ranged from 0.1 mL for mice (ca. 25-40 g) to 0.6 mL for rats (ca. 120-160 g).

This biodistribution study of three DDG analogs comprised 36 animals, with five tissues per animal to process and analyze, i.e., 180 water-soluble samples and 180 lipid samples.

In mice injected with a bolus dose of 43-45 mg/kg [D4]DDG (dose equivalent to non-labeled 2DG), 0.23 mM was detected in the liver (n=2), 1.79 mM in the kidneys (n=1), and 3.14 mM in the heart (n=2).

In rats injected with a bolus dose of 37-55 mg/kg [D4]DDG (dose equivalent to non-labeled 2DG), we found 1.60 mM in the brain (n=6), 2.70 mM in the heart (n=2), and 0.08 mM in the muscle (n=6).

In mice injected with a bolus dose of 169-182 mg/kg [D2]DDG (dose equivalent to non-labeled 2DG), we found 1.19 mM in the brain (n=2) and 0.4 mM in the liver (n=1).

A typical ²H-NMR spectrum from a rat brain is shown in FIG. 7. The rat was injected with 45 mg/kg of [D4]DDG and was awake for 36 min between the injection and sacrifice. The signal on the left is due to the natural abundance of deuterium in water (HDO). It is truncated for better visibility of the signal on the right, which is due to the deuterons of [D4]DDG.

While [18F]-fluoro-2-deoxy-D-glucose accumulates at very high levels in the human brain, the biodistribution of the deuterated DDGs appears to occur with a different pattern. This may be due to changes in the doses used in this study as compared with PET studies.

1. A method comprising acquiring at least one magnetic resonance (MR)²H spectrum and/or at least one MR ²H image from a subject, wherein the subject has been administered with a 2-deoxy-D-glucose (2DG) compound comprising one or more isotopically substituted hydrogen atoms.

2. The method of claim 1, wherein said at least one ²H image is acquired at an acquisition time of at most about 5 min.

3. The method of claim 1, wherein said at least one ²H MR image is acquired from the subject with a spatial resolution of between about 2 mm and about 20 mm.

4. The method of claim 1, wherein said at least one ²H MR image is acquired using a pulse sequence that has a component of inversion recovery or a saturation component.

5. The method of claim 1, wherein the 2DG compound is at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof.

6. The method of claim 5, wherein the 2DG compound is one or more of [²H₈]2-deoxy-D-glucose, [²H₂]2-deoxy-D-glucose and [²H₄]2-deoxy-D-glucose, optionally wherein the compound is [3,4,6,6-²H4]2-deoxy-D-glucose.

7. The method of claim 1, wherein the 2DG compound is represented by Formula (I): 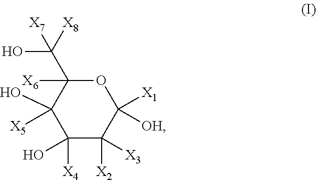
or a pharmaceutically acceptable salt, solvate, hydrate, any tautomer thereof, any isomer thereof, any metabolite thereof or physiologically functional derivative thereof, wherein at least one of X₁, X₂, X₃, X₄, X₅, X₆, X₇, X₈ is ²H (D), wherein (i) one of X₁, X₂, X₃, X₄, X₅, X₆, X₇, and X₈ is ²H, (ii) two of X₁, X₂, X₃, X₄, X₅, X₆, X₇, and X₈ is ²H, (iii) three of X₁, X₂, X₃, X₄, X₅, X₆, X₇, and X₈ is ²H, (iv) four of X₁, X₂, X₃, X₄, X₅, X₆, X₇, and X₈ is ²H, (v) five of X₁, X₂, X₃, X₄, X₅, X₆, X₇, and X₈ is ²H, (vi) six of X₁, X₂, X₃, X₄, X₅, X₆, X₇, and X₈ is ²H, (vii) seven of X₁, X₂, X₃, X₄, X₅, X₆, X₇, and X₈ is ²H, (viii) eight of X₁, X₂, X₃, X₄, X₅, X₆, X₇, and X₈ is ²H or a combination thereof.

8. The method of claim 7, wherein said 2DG compound is represented by one or more of:
(a) Formula (IV): 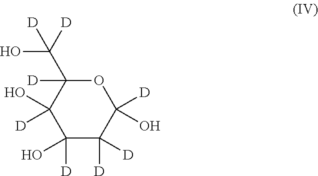 [SMILES: [2H]C([2H])(O)C1([2H])OC([2H])(O)C([2H])([2H])C([2H])(O)C1([2H])O]
or a pharmaceutically acceptable salt, solvate, hydrate, any isomer thereof, any metabolite thereof or physiologically functional derivative thereof,
(b) Formula (VI): 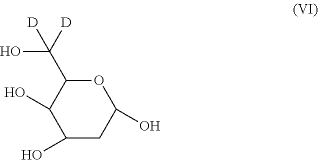 [SMILES: [2H]C([2H])(O)C1OC(O)CC(O)C1O]
or a pharmaceutically acceptable salt, solvate, hydrate, any isomer thereof, any tautomer thereof, any metabolite thereof or physiologically functional derivative thereof,
(c) Formula (VIII): 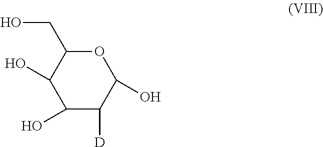 [SMILES: [2H]C1C(O)OC(CO)C(O)C1O]
or a pharmaceutically acceptable salt, solvate, hydrate, any isomer thereof, any tautomer thereof, any metabolite thereof or physiologically functional derivative thereof,
(d) Formula (X): 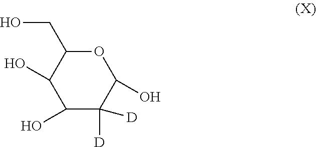 [SMILES: [2H]C1([2H])C(O)OC(CO)C(O)C1O]
or a pharmaceutically acceptable salt, solvate, hydrate, any isomer thereof, any tautomer thereof, any metabolite thereof or physiologically functional derivative thereof,
(e) Formula (XX): 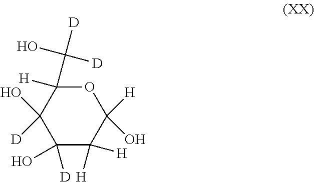 [SMILES: [2H]C([2H])(O)C1OC(O)CC([2H])(O)C1([2H])O]
or a pharmaceutically acceptable salt, solvate, hydrate, any isomer thereof, any tautomer thereof, any metabolite thereof or physiologically functional derivative thereof, or (f) any combination thereof.

9. The method of claim 1, wherein said compound has a T₁ relaxation of a ²H nucleus of between about 5 milliseconds to about 200 milliseconds.

10. The method of claim 1, further comprising detecting in a tissue of said subject at least one metabolite of said compound, wherein said metabolite is a deuterium-labeled 2-deoxy-D-glucose-6-phosphate.

11. The method of claim 10, wherein said metabolite is one of [D]2-deoxy-D-glucose-6-phosphate, [D₂]2-deoxy-D-glucose-6-phosphate, [D₃]2-deoxy-D-glucose-6-phosphate, [D₄]2-deoxy-D-glucose-6-phosphate, [D₅]2-deoxy-D-glucose-6-phosphate, [D₆]2-deoxy-D-glucose-6-phosphate, [D₇]2-deoxy-D-glucose-6-phosphate, [D₈]2-deoxy-D-glucose-6-phosphate or a combination thereof.

12. The method of claim 1, for diagnosing a disease in said subject, monitoring a disease state in said subject and/or determining a site of a disease in said subject.

13. The method of claim 12, wherein the disease is a proliferative disorder.

14. The method of claim 1, for distinguishing between healthy and abnormal tissues or organs and/or distinguishing or differentiating between malignant and benign tumors.

15. The method of claim 1 for imaging a subject, comprising monitoring a signal from a subject using an MR imaging method, the subject having been administered at least one 2DG compound to thereby acquire at least one MR image, wherein said 2DG compound is deuterium-labeled 2DG.

16. A method for diagnosis of a disease or condition in a subject, said method comprising (i) administrating a diagnostically effective amount of at least one 2DG compound to the subject, (ii) obtaining an ²H MR image and/or ²H MR spectrum of said subject or of a body region of the subject; (iii) identifying body regions in which said compound, or any metabolite thereof has been localized, wherein said 2DG compound is a deuterium-labeled 2DG; thereby enabling the diagnosis of a disease or condition in a subject according to the localization of the 2DG compound or its metabolites.

17. The method of claim 16 for determining the severity of a disease or condition in the subject, the method comprising (i) imaging and/or acquiring at least one spectrum the subject's body or region thereof to obtain at least one imaging parameter, and (ii) comparing said at least one imaging parameter to at least one parameter obtained from said subject at the onset of treatment or prior to treatment commencement, and thereby determining the severity of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), Formula (XX), Formula (XXI), or a pharmaceutically acceptable salt, solvate, hydrate, any isomer thereof, any metabolite thereof or physiologically functional derivative thereof, or (f) a combination thereof.

18. The method of claim 16 for determining the effectiveness of a therapeutic treatment of a disease or disorder in a subject, the method comprising (i) imaging the subject's body or region thereof and thereby obtaining at least one imaging parameter, and (ii) comparing said at least one imaging parameter to at least one parameter obtained from said subject at the onset of treatment or prior to treatment commencement; and thereby determining the effectiveness of the therapeutic treatment of the disease or disorder in the subject, wherein said compound is any one of (a) a deuterium labeled 2DG, (b) at least one of [²H]2-deoxy-D-glucose, [²H₂]2-deoxy-D-glucose, [²H₃]2-deoxy-D-glucose, [²H₄]2-deoxy-D-glucose, [²H₅]2-deoxy-D-glucose, [²H₆]2-deoxy-D-glucose, [²H₇]2-deoxy-D-glucose, [²H₈]2-deoxy-D-glucose or a combination thereof, (c) [²H₈]2-deoxy-D-glucose, (d) represented by Formula (I) or Formula (I′) and/or Formula (II), and/or Formula (II′), and/or Formula (III), and/or Formula (III′), or a pharmaceutically acceptable salt, solvate, hydrate, any isomer thereof, any metabolite thereof or physiologically functional derivative thereof, wherein at least one of X₁, X₂, X₃, X₄, X₅, X₆, X₇, X₈ is ²H (D), (e) represented by Formula (IV)-Formula (XI), Formula (XX), Formula (XXI), or a pharmaceutically acceptable salt, solvate, hydrate, any isomer thereof, any metabolite thereof or physiologically functional derivative thereof, or (f) a combination thereof.

19. A method of treating, preventing, inhibiting, reducing, eliminating, protecting or delaying the onset of at least one disease or condition in a subject in need thereof, said method comprising: (i) administrating a diagnostically effective amount of at least one 2DG compound to the subject, (ii) obtaining an ²H MR image and/or ²H MR spectrum of said subject or of a body region of the subject; (iii) identifying body regions in which said compound, or any metabolite thereof has been localized, wherein said 2DG compound is a deuterium-labeled 2DG; and (iv) administering to the subject a treatment regimen based on said disease or condition thereby treating said disease or condition.
